(19) United States
(12) Patent Application Publication
(10) Pub. No.: US 2025/0155458 A1
(43) Pub. Date: May 15, 2025

(54) UTILIZING TRACE AMOUNTS OF A SUSPECTED TARGET MOLECULE AS A CATALYST IN DETECTION REACTIONS

(71) Applicant: Lawrence Livermore National Security, LLC, Livermore, CA (US)

(72) Inventors: Carlos A. Valdez, San Ramon, CA (US); Mark Miles Hart, Livermore, CA (US); Mathew Gerald Lyman, Brentwood, CA (US)

(21) Appl. No.: 18/388,766

(22) Filed: Nov. 10, 2023

(51) Int. Cl. G01N 33/94; G01N 21/64

(52) U.S. Cl. CPC G01N 33/9486; G01N 21/6428; G01N 33/94; G01N 33/946; G01N 2021/6439

(57) ABSTRACT

A method for detecting a suspected target molecule includes obtaining a reaction mixture that is not reactive at ambient temperature, the reaction mixture comprising at least two reactants, adding the suspected target molecule to the reaction mixture for causing a reaction involving the reactants in the reaction mixture, and detecting presence of a product formed from the reactants in response to the addition of the suspected target molecule. The suspected target molecule acts as a catalyst for the reaction, where the suspected target molecule includes a tertiary amine.

This invention was made with Government support under Contract No. DE-AC52-07NA27344 awarded by the United States Department of Energy. The Government has certain rights in the invention.

### FIELD OF THE INVENTION

The present invention relates to detecting suspected target molecules, and more particularly, this invention relates to utilizing a suspected target molecule as a catalyst in detection reactions at ambient conditions.

### BACKGROUND

Detection of various narcotics and controlled substances, such as opioids (natural, semi-synthetic and synthetic), cocaine, LSD, etc. relies on specific instrumentation designed for detecting stoichiometric amounts of the suspected target drugs. Other detection approaches include the use of colorimetric reactions between the suspected target drug and dyes that provide visible color change in a matter of minutes, but in many instances the methods require stoichiometric or excess amounts of the suspected target drug. Moreover, frequently further analysis is needed to confirm the identity of the suspected target drug.

### Opioids and Opioid Derivatives

Opioids are substances that act on opioid receptors to produce morphine-like effects. Morphine is a natural opioid extracted from the opium poppy. Morphine has been the gold standard in pain management in the medical field for decades and its impact in this area is unmatched by other drugs. Due to its euphoric effects brought about by its use and its effects in the central nervous system, patients can develop heavy dependency on the drug. Morphine is also used as a starting material for the large-scale production of one of the most common and devastating drugs making its way across the US border, heroin. The conversion of morphine into heroin is simple and can be accomplished by treating the opium extracts with acetic anhydride and using a stepwise acidic and basic adjustments in a well-established process. The final result is multi-kilogram quantities of heroin.

Heroin is a semi-synthetic opioid that can be made from the natural opioid morphine obtained in large quantities by efficient extraction from the opium poppy. Heroin has been used as a recreational drug for decades and continues to pose a threat to civilian communities across the United States and other countries, increasing the complexity and aggravating further the current opioid crisis. In addition to this role, heroin particularly in parts of the world like Southeast Asia, the Middle East and South America is used as a common unit for the acquisition of capital to purchase weapons and fund other illegal narcotics' production and distribution. Heroin still remains a central piece in the overall monetary influx for drug cartels and even governments in these parts of the world. Its trafficking into the United States and other nations is a real, ongoing problem and as such the development of technologies for its effective and rapid detection is of paramount importance if an effective program combating its trade and use is to be implemented by a nation.

Currently, morphine, heroin, etc. are currently detected using portable Raman spectrophotometers, but this instrumentation needs the material in (relatively) pure form as its efficacy relies on spectral comparison of the sample with that of its internal library.

For morphine, an alternate but more inexpensive option is the use of colorimetric reactions between morphine, heroin, etc. with dyes that can provide a visible color change in a matter of minutes. As illustrated in FIG. 1A, one method is known as the idPAD; it is a piece of paper that has been laced in several lanes with different reagents. Morphine is applied (˜20 mgs) across the bottom of the paper (part (a)) and then “developed” with a solvent front so that the drug gets carried up the paper via capillary action. As the drug interacts with reagents in each lane, it will give a characteristic color. Part (b) illustrates the interaction of morphine molecules with reagents in lanes A-C, and part (c) illustrates the interaction of methamphetamine with reagents in lanes A-C. This technology provides a means to alert the user that morphine may be present in a mixture, but further testing is still required using conventional laboratory analytical techniques. The idPAD system is also used for the detection heroin following a similar protocol.

The various reagents may give false positives, and as such, several different reagents are included in the idPAD, so that a “fingerprint” pattern is obtained for a given drug. Colorimetric methods, although visible with the naked eye, use stoichiometric or excess amounts of the morphine to produce a signal that is deemed diagnostic but not fully confirmatory. The confirmation is usually done afterwards using laboratory instrumentation for unambiguous identification, such as Gas Chromatography-Mass Spectrometry (GC-MS) and/or Liquid Chromatography-Mass Spectrometry (LC-MS).

There is a need for rapid identification of morphine, preferably via a visual test at the site of exposure.

Codeine, hydrocodone, and oxycodone are opioids used in the treatment of pain. These opioids are highly addictive causing physical as well as psychological dependence, in similar fashion to morphine and other related opioids. Common side effects associated with the consumption of these opioids include euphoria, delirium and hallucinations. Hydrocodone can be given in combination with other less potent pain medications such as acetaminophen and ibuprofen when the need for alleviating severe pain arises. One of the main reasons for the existence of such pain relieving cocktails is the addictive properties of hydrocodone, which are observed in patients who only depend on the opioid for pain management.

Consumers of hydrocodone become highly dependent with increased tolerance to the drug, thus increasing the chances of their death by overdosing. In 2011, hydrocodone products were involved in around 100,000 abuse-related emergency department visits in the United States, more than double the number in 2004. Moreover, clandestine laboratories are known to use codeine in the synthesis of morphine and other related alkaloids.

In situations where analysis of codeine, hydrocodone, or oxycodone need to be performed, the detection of the opiate after the sample has been collected (e.g., pure form or as part of a biological collection like urine or blood) depends heavily on laboratory instrumentation such as GC-MS and/or LC-MS. Currently no direct colorimetric detection specifically for codeine, hydrocodone, or oxycodone exists but it can be anticipated that colorimetric techniques used for other alkaloids/drugs may work to some extent in this area. In instances where the opiate is found in the environment, its detection is usually performed by the same screening test kits used for the detection of other opiates such as morphine and heroin.

However, these tests are not specific and only rapid method to determine the potential presence of the toxic substance in the environment (e.g., during an emergency crisis situation, drug seizure, etc.) and thus merely provides an alarm to first responders, law enforcement personnel, etc. These procedures apply to the analysis of pills or other forms of the drug that can be easily and rapidly screened by law enforcement, field analysts, etc.

The massive influx of synthetic opioids such as fentanyls, fentanyl analogs, and other opioids into the United States has been recently highlighted by congressional hearings and reports from the Drug Enforcement Administration. Fentanyls are used in the medical community as an analgesic, but also ingested illicitly and accidentally (e.g., laced prescription pills). Detection of synthetic opioids such as fentanyl/fentanyl analogs currently include available commercial colorimetric detection methods such as fentanyl test strips that make use of an antibody-antigen approach to facilitate the detection of these substances. This technology provides a means to alert the user that a fentanyl may be present in a mixture, but further testing is still required using conventional laboratory analytical techniques.

### Tropane Alkaloids

Scopolamine is a natural alkaloid belonging to the tropane family of alkaloids. It is one of the active ingredients found in plants belonging to the nightshade family (i.e., Solanaceae family), and it is a potent antimuscarinic drug that is used in the clinic to treat mainly motion sickness and postoperative nausea. Scopolamine is a psychoactive drug (known as a deliriant) and it can cause severe hallucinations in individuals consuming it. It has been used as a mind-altering drug for nefarious purposes which has resulted in Scopolamine being known criminal circles as Devil's Breath, Dragon's breath and commonly known in Latin America as Burundanga.

The drug is commonly blown into the victim's face and once inhaled, Scopolamine rapidly begins to affect the central nervous system completely subduing the victim. Scopolamine is used for what is called chemical submission. Many of the victims have been robbed or raped without being able to fight or resist, as the drug causes a total loss of willpower, followed by an absence of memory of the events. The recreational use of this drug is well recognized as well as its use to subdue victims for criminal purposes (e.g., sexual assaults).

It is for these reasons that drug agencies and research groups (both industry and academia), have pursued the development of detection methods for this drug that does not involve laboratory equipment such as GC-MS or LC-MS. The main reason for this is that a rapid test is needed by law enforcement that can provide a rapid and tentative identification. For detection of scopolamine, as illustrated in FIG. 1B, a similar type option is the use of colorimetric reactions between scopolamine with dyes/antibody combos in what is known as the NarcoCheck. The NarcoCheck SCOP test strip which is an immunoassay-based test strip for urine samples designed for a victim who has been exposed to the drug. The test strip includes antibodies having specificity for scopolamine. Briefly, the strip is dipped in a urine sample and as the liquid makes its way up the strip via capillary action, any Scopolamine present in it will interact with an immobilized antibody on the strip (FIG. 1B). The level of detection is down to ˜500 ng/ml of the drug in urine, thus making the strip a very sensitive detecting method for this dangerous alkaloid.

Atropine is a natural alkaloid that is produced by the plant Atropa belladonna also known as deadly nightshade. It can be found in others belonging to the same family such as mandrake and Jimsom weed. Atropine has been used as an anticholinergic drug and most notably to combat the effects of nerve agents where it has been used in conjunction with oxime reactivators (e.g., Mark I Nerve Agent antidote injection kit) to treat affected individuals, both military and civilians alike. Atropine is cheap and can be obtained in large quantities from the plants that produce it. Even though its medical uses are well recognized, the alkaloid can be toxic leading to death by overdose.

For detection of atropine, the current methodology available includes commercial colorimetric detection method that involves the reaction of atropine with cobalt isocyanate and ferric hydroxamate as illustrated in FIG. 1C. These methods although producing visible coloration detected with the naked eye, however the primary limitation is the use of stoichiometric or excess amounts of the atropine to produce a signal that is deemed diagnostic not fully confirmatory. The confirmation is usually done afterwards using conventional laboratory analytical techniques such as GC-MS and LC-MS. This technology provides a means to alert the user that atropine may be present in a mixture, but further testing is still required.

Cocaine is a natural alkaloid that is produced by the coca plant Erythroxylum coca. Coca, the raw material for cocaine, is grown mainly in the Andean region of South America. Cocaine production constitutes a threat to U.S. security and the well-being of citizens. Due to its euphoric effects, users develop dependency on the drug (and in many cases die via overdose). According to U.S. Government estimates, 95% percent of the cocaine entering the United States originates in Colombia; Peru and Bolivia are other Andean nations that cultivate significant levels of coca.

Therefore, one way to combat the traffic of the drug across the United States border is by detecting it with either colorimetric tests or more advanced analytical techniques. In the case of colorimetric tests, it is important to know that these tests are preliminary and not a definite identification of the alkaloid. However, these tests can be used to quickly assess whether a sample may potentially be cocaine; further tests involving other analytical techniques like Raman spectroscopy or GC-MS can then be used to unambiguously identify the material.

As illustrated in FIG. 1D, detection of cocaine include colorimetric detection methods that involve the interactions between the alkaloid and cobalt thiocyanate (blue coloration indicating presence of cocaine) (part (a)), the modified cobalt thiocyanate method (Scott's test) (blue color first, then pink signals the presence of cocaine) (part (b)), and one that involves odor identification, the methyl benzoate test, part (c). The primary limitation of these methods is the use of stoichiometric or excess amounts of the cocaine (˜20-50 mgs per test) to produce a signal that is deemed diagnostic. Therefore, they are not very useful when low levels of the alkaloid are present (<1 mg). These technologies provide a means to alert the user that cocaine may be present in a mixture, but further testing is still required using conventional laboratory analytical techniques. The confirmation is usually done afterwards using benchtop techniques such as GCMS and LC-MS.

### Other Narcotic Drugs

Lysergic acid diethylamide (LSD) is a synthetic powerful psychedelic drug. LSD can be habit forming and some of its effects include intensified thoughts, emotions, and sensory perception. At sufficiently high doses, the drug manifests primarily mental, visual, as well as auditory hallucinations. LSD is a solid compound, typically in the form of a powder or a crystalline material that can be dissolved in common solvents like an alcohol (e.g., ethanol) or distilled water, in order to accurately (or as accurately as possible) load a given dose of the drug onto small pieces of blotter paper called “tabs”. LSD is typically either swallowed or held under the tongue where it makes its way into the bloodstream. LSD is considered to be non-addictive with low potential for abuse, however, its synthesis, distribution and consumption remains illegal in the US.

From a detection perspective, LSD, like other drugs, can be detected by portable Raman spectrophotometers, but this instrumentation needs the material in (relatively) pure form as its efficacy relies on spectral comparison of the sample with that of its internal library. As illustrated in FIG. 1E, currently, colorimetric methods of detection of LSD exist that are based on the change of color of Ehrlich's reagent (part (a)) turning purple upon contact with the drug and a NarcoCheck detection strip (part (b)). These tests are not specific but provide a rapid and quick way to determine the potential presence of this drug in the environment (e.g., during a drug seizure) and thus efficiently alarming first responders or law enforcement personnel who could be inadvertently exposed to the drug.

Pink Sunshine, or 1-propanoyl-lysergic acid diethylamide (1P-LSD), is a powerful psychedelic drug. 1P-LSD can be habit forming and some of its effects include intensified thoughts, emotions, and sensory perception similar to those arising from LSD intake. 1P-LSD is considered a designer drug and a functional analog of LSD. At sufficiently high doses, the drug manifests primarily mental, visual, as well as auditory hallucinations. 1P-LSD is a solid compound, typically in the form of a powder or a crystalline material that can be dissolved in common solvents like an alcohol (e.g., ethanol) or distilled water, in order to accurately (or as accurately as possible) load a given dose of the drug onto small pieces of blotter paper called “tabs”.

A study in 2020 found that IV administration of 1P-LSD has a somewhat shorter duration than LSD in humans and that the drug is present for a very short time (t=4 hr) in plasma prior to being completely metabolized to LSD. 1P-LSD is typically either swallowed or held under the tongue where it makes its way into the bloodstream. Unlike LSD, 1P-LSD is non-scheduled unless it is sold for human consumption as a closely related analog to LSD. Currently, colorimetric methods for the detection of 1PLSD do not exist because the structure of the designer drug IP-LSD (i.e., indole ring) is not similar to LSD and behaves differently. Therefore, accurate detection and analysis of the presence of IP-LSD relies on conventional laboratory equipment. However, detection by portable Raman spectrophotometers requires the suspected material to be presence in relatively pure form since the efficacy of the methodology depends on a spectral comparison of the sample with an internal library. Currently, 1P-LSD is detected separately at a laboratory after a sample has been collected (e.g., pure form or as part of a biological collection like urine or blood) using instrumentation, such as GC-MS or LC-MS).

Thus, there is an essential need for methods for the rapid identification of narcotics such as opioids and synthetic opioid derivatives, tropane alkaloids, and other narcotics via a visual test, more advanced analytical equipment (GC-MS or LC-MS), etc. in the environment. In narcotic drug contamination crisis situations, the use of conventional field kits, conventional laboratory detection methods, etc. would be impractical for readily identifying contaminated surfaces and equipment. In particular, advanced quick detection methods for identification of narcotic drugs at contaminated labs and surrounding areas are needed for efficient and safe containment of the contaminating drugs.

### SUMMARY

According to one embodiment, a method for detecting a suspected target molecule includes obtaining a reaction mixture that is not reactive at ambient temperature, the reaction mixture comprising at least two reactants, adding the suspected target molecule to the reaction mixture for causing a reaction involving the reactants in the reaction mixture, and detecting presence of a product formed from the reactants in response to the addition of the suspected target molecule. The suspected target molecule acts as a catalyst for the reaction, where the suspected target molecule includes a tertiary amine.

According to another embodiment, a product for detecting a suspected target molecule includes a substrate and a reaction mixture coupled to the substrate. The reaction mixture includes at least two reactants, where the reaction mixture is not reactive at ambient temperature. Moreover, the reaction mixture is configured to be reactive upon exposure thereof to a suspected target molecule that acts a catalyst, thereby causing the reaction mixture to react and form a product.

According to yet another embodiment, a solution for detecting suspected target molecule includes a reaction mixture having at least two reactants, and a solvent, where the reaction mixture is not reactive in the solvent at ambient temperature. The reaction mixture is configured to be reactive upon exposure to a suspected target molecule that acts as a catalyst, thereby causing the reaction mixture to react and form a product.

Other aspects and advantages of the present invention will become apparent from the following detailed description, which, when taken in conjunction with the drawings, illustrate by way of example the principles of the invention.

### BRIEF DESCRIPTION OF THE DRAWINGS

FIG. 1A is an image of an idPAD colorimetric detection kit for the detection of morphine.

FIG. 1B is an image of a NarcoCheck SCOP colorimetric test used for the detection of scopolamine.

FIG. 1C a schematic drawing of the reaction of atropine with cobalt isocyanate and ferric hydroxamate for the detection of atropine.

FIG. 1D is a schematic drawing of various colorimetric detection methods for detection of cocaine. Part (a) illustrates the interaction between cocaine and cobalt thiocyanate, part (b) illustrates Scott's test, and part (c) illustrate the methyl benzoate test.

FIG. 1E are images of colorimetric detection methods for detection of LSD. Part (a) Ehrlich's reagent, and part (b) NarcoCheck detection strip.

FIG. 2A depicts schematic drawings of opioid and synthetic opioids molecules.

FIG. 2B depicts schematic drawings of fentanyl and fentanyl analogs. Part (a) depicts the molecule structures, and part (b) depicts the affinity of fentanyl molecules for the biological target.

FIG. 2C depicts schematic drawings of tropane alkaloid molecules.

FIG. 2D depicts schematic drawings of LSD and LSD derivatives.

FIG. 3 is a flow chart of a method for detecting a suspected target molecule, according to one embodiment.

FIG. 4 includes schematic drawings of a detection method using a suspected target molecule as a catalyst in a reaction mixture of a thiol and an isocyanate, according to one embodiment. Part (a) depicts the reaction, part (b) depicts a generalized reaction, and part (c) depicts a series of suspected target molecules.

FIG. 5 includes schematic drawings of a detection method using a suspected target molecule as a catalyst in a reaction mixture of a nitroalkane and an isocyanate, according to one embodiment. Part (a) depicts the reaction, part (b) depicts a generalized reaction, and part (c) depicts a series of suspected target molecules.

FIG. 6 includes schematic drawings of a detection method using a suspected target molecule as a catalyst in a reaction mixture of an alcohol and an isocyanate, according to one embodiment. Part (a) depicts the reaction, part (b) depicts a generalized reaction of an alcohol and an isothiocyanate, parts (c) and (d) depict a FRET approach that includes the alcohol having an acceptor dye and the isocyanate having a donor dye, and part (e) depicts the reaction where the fluorescent dye is quenched by another dye.

FIG. 7 includes schematic drawings of formation of a polymer using a polymerization detection method that uses a suspected target molecule as a catalyst in a reaction mixture, according to one embodiment. Part (a) depicts a reaction, part (b) depicts a generalized reaction, and part (c) depicts a series of suspected target molecules.

FIG. 8 is a series of schematic drawings that depict a polymerization reaction using a suspected target molecule as a catalyst in a reaction that includes fluorescent and phosphorescent polyurethane, according to one embodiment.

FIG. 9 includes schematic drawings of a fluorescence detection system using a suspected target molecule as a catalyst for the reaction, according to one embodiment. Part (a) depicts the reaction, and part (b) depicts a series of suspected target molecules.

FIG. 10 includes schematic drawing of a fluorescence detection system using a modified Hinsberg reaction, according to one embodiment. Part (a) depicts the reaction, and part (b) depicts a series of suspected target molecules.

FIG. 11 is an example of a colorimetric detection of atropine, according to one approach. Part (a) is a schematic drawing of the reaction, part (b) is a schematic drawing of a generalized reaction, and part (c) is a series of images of the colorimetric test of atropine as a catalyst in a reaction mixture with a nitromethane solvent and a diethyl ether solvent.

FIG. 12 is an example of a colorimetric detection of acetylfentanyl, according to one approach. Part (a) is a schematic drawing of the reaction, and part (b) is an image of the colorimetric test of varying amounts of acetylfentanyl in a reaction mixture.

FIG. 13 is an example of a polymerization reaction using acetylfentanyl as a catalyst, according to one approach. Part (a) is a schematic drawing of the reaction, part (b) is an image of the vials containing the reactants with different solvents, part (c) is an image of the vials including the reactants and the acetylfentanyl as a catalyst, and part (d) is a vial including the reactants only as a control.

FIG. 14A is an example of a direct fluorescent reaction using fentanyl as a catalyst, according to one approach. Part (a) is a schematic drawing of the reaction, and part (b) is an image of the vials containing the reaction mixture without fentanyl and with fentanyl.

FIG. 14B is an example of a direct fluorescent modified Hinsberg reaction using atropine or acetylfentanyl as a catalyst, according to one approach. Part (a) is a schematic drawing of the reaction, and part (b) are images of the vials containing the reaction mixture with varying concentrations of atropine (left image) and varying concentration of acetylfentanyl (right image).

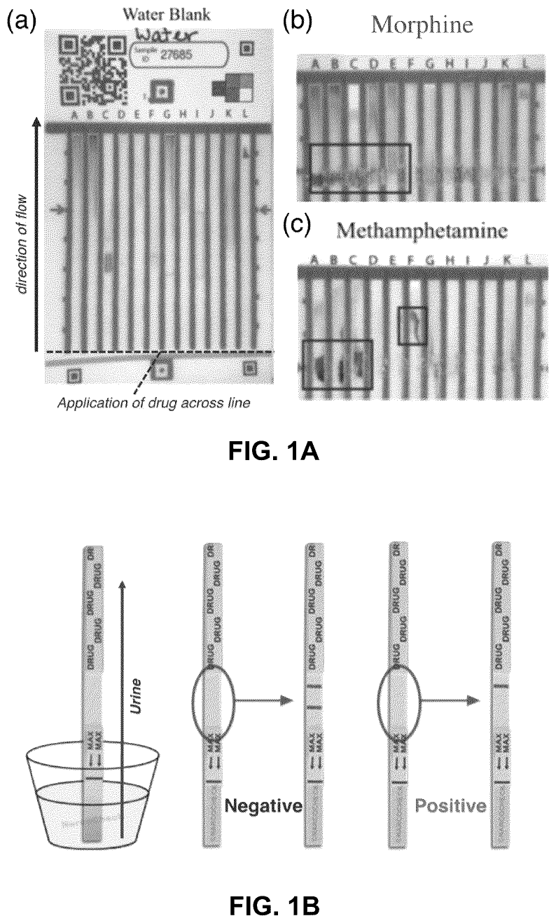

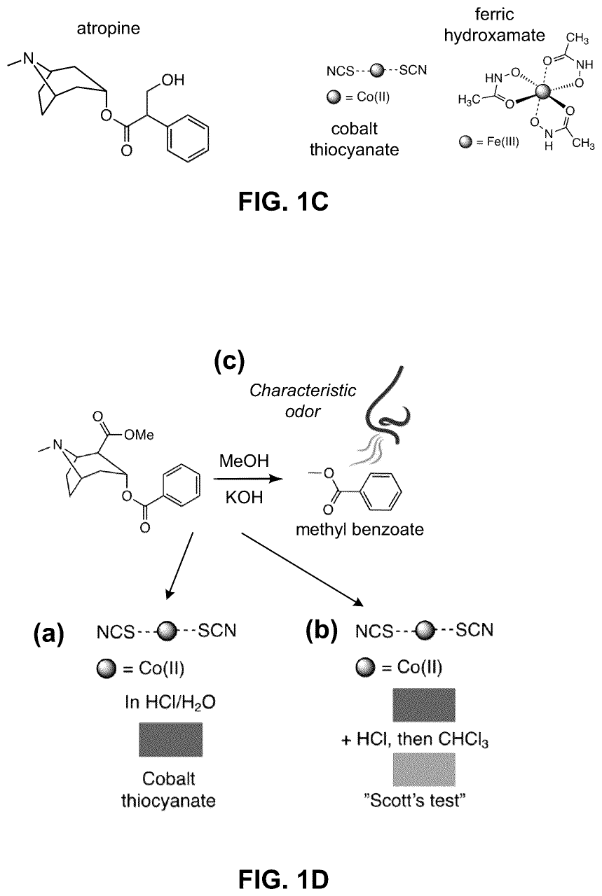

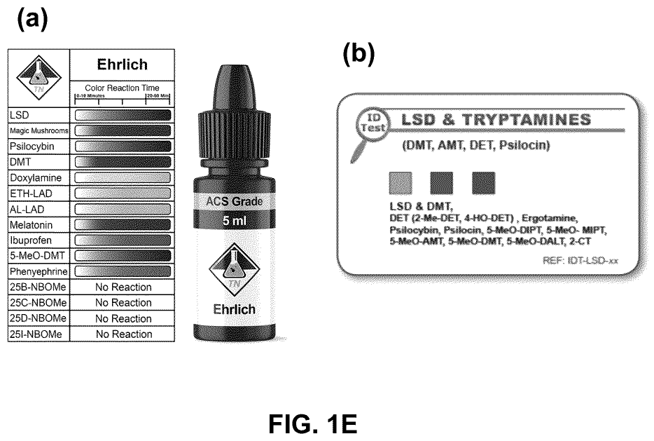

### DETAILED DESCRIPTION

The following description is made for the purpose of illustrating the general principles of the present invention and is not meant to limit the inventive concepts claimed herein. Further, particular features described herein can be used in combination with other described features in each of the various possible combinations and permutations.

Unless otherwise specifically defined herein, all terms are to be given their broadest possible interpretation including meanings implied from the specification as well as meanings understood by those skilled in the art and/or as defined in dictionaries, treatises, etc.

It must also be noted that, as used in the specification and the appended claims, the singular forms “a,” “an” and “the” include plural referents unless otherwise specified.

As also used herein, the term “about” denotes an interval of accuracy that ensures the technical effect of the feature in question. In various approaches, the term “about” when combined with a value, refers to plus and minus 10% of the reference value. For example, a thickness of about 10 nm refers to a thickness of 10 nm±1 nm, a temperature of about 50° C. refers to a temperature of 50° C.±5° C., etc.

It is noted that room temperature may be defined as a temperature in a range of about 20° C. to about 25° C. Ambient room temperature is an actual temperature, measured by a thermometer of the air, medium, surroundings, etc. in any particular environment.

It is also noted that, as used in the specification and the appended claims, wt. % is defined as the percentage of weight of a particular component is to the total weight/mass of the mixture. Vol. % is defined as the percentage of volume of a particular compound to the total volume of the mixture or compound. Mol. % is defined as the percentage of moles of a particular component to the total moles of the mixture or compound. Atomic % (at. %) is defined as a percentage of one type of atom relative to the total number of atoms of a compound.

Unless expressly defined otherwise herein, each component listed in a particular approach may be present in an effective amount. An effective amount of a component means that enough of the component is present to result in a discernable change in a target characteristic of the ink, printed structure, and/or final product in which the component is present, and preferably results in a change of the characteristic to within a desired range. One skilled in the art, now armed with the teachings herein, would be able to readily determine an effective amount of a particular component without having to resort to undue experimentation.

The following description discloses several preferred embodiments of utilizing a suspected target molecule as a catalyst in detection systems and/or related systems and methods.

In one general embodiment, a method for detecting a suspected target molecule includes obtaining a reaction mixture that is not reactive at ambient temperature, the reaction mixture comprising at least two reactants, adding the suspected target molecule to the reaction mixture for causing a reaction involving the reactants in the reaction mixture, and detecting presence of a product formed from the reactants in response to the addition of the suspected target molecule. The suspected target molecule acts as a catalyst for the reaction, where the suspected target molecule includes a tertiary amine.

In another general embodiment, a product for detecting a suspected target molecule includes a substrate and a reaction mixture coupled to the substrate. The reaction mixture includes at least two reactants, where the reaction mixture is not reactive at ambient temperature. Moreover, the reaction mixture is configured to be reactive upon exposure thereof to a suspected target molecule that acts a catalyst, thereby causing the reaction mixture to react and form a product.

In yet another general embodiment, a solution for detecting suspected target molecule includes a reaction mixture having at least two reactants, and a solvent, where the reaction mixture is not reactive in the solvent at ambient temperature. The reaction mixture is configured to be reactive upon exposure to a suspected target molecule that acts as a catalyst, thereby causing the reaction mixture to react and form a product.

A list of acronyms used in the description is provided below.
3° amine tertiary amine
BBB blood-brain barrier
C Celsius
EI-GC-MS Electron Impact Gas Chromatography-Mass Spectrometry
FITC fluorescein isothiocyanate
FRET Förster resonance energy transfer
GC-MS Gas Chromatography-Mass Spectrometry
LC-MS Liquid Chromatography-Mass Spectrometry
mol. % mole percent
MOR μ-opioid receptor
UV ultraviolet
wt. % weight percent

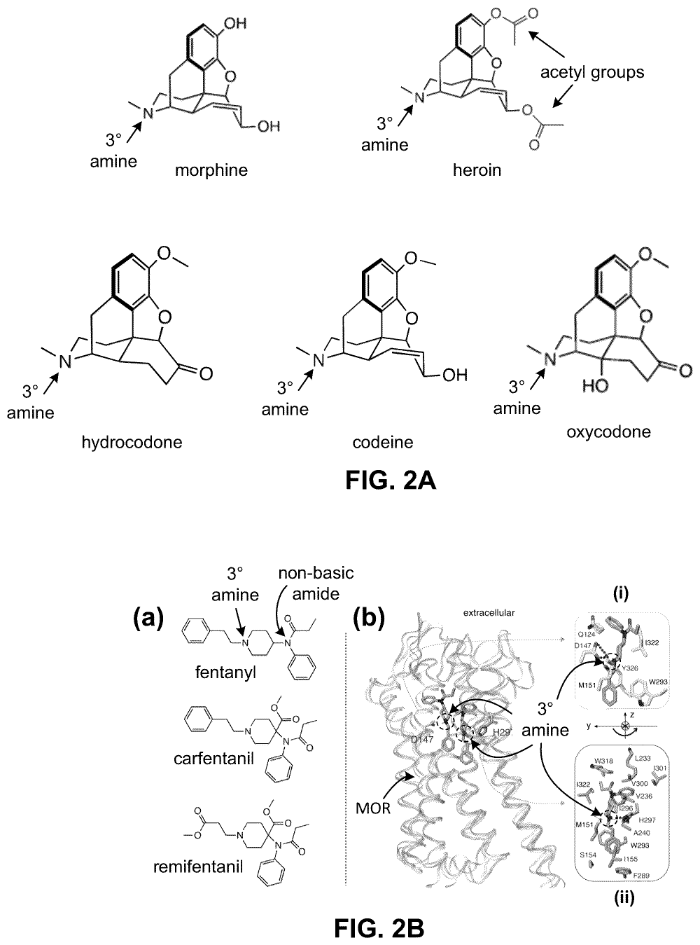

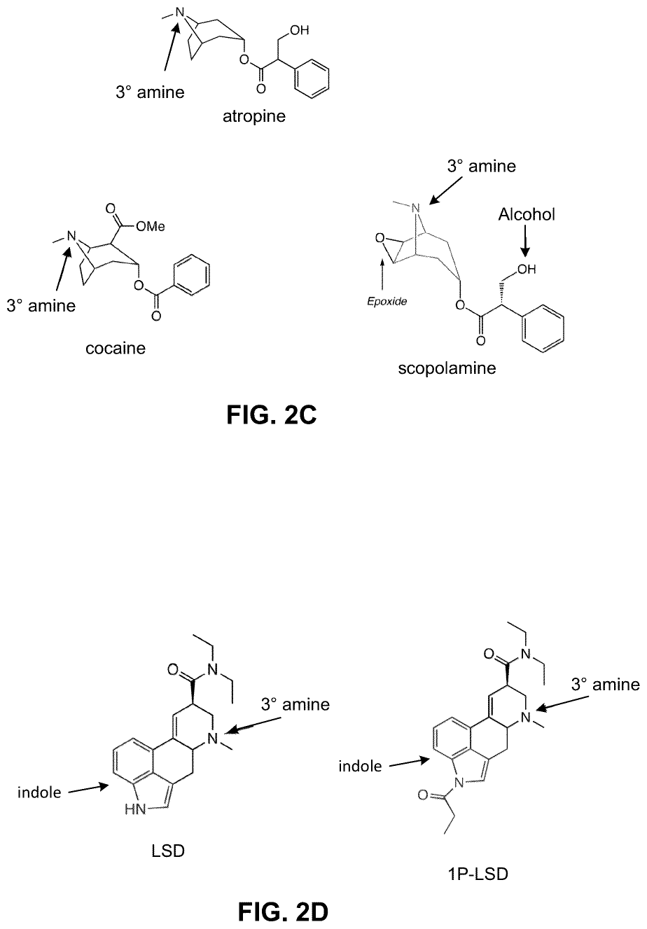

The mode of action of opioids, synthetic opioids, tropane alkaloids, and other narcotic drugs involves binding the key receptors in the brain leading to altering effects on the nervous system, respiratory system, etc. For instance, as illustrated in FIG. 2A, many opioids share similar aspects of the molecule structure.

Various embodiments described herein allow a suspected target molecule to be detected via a signaling event that can be a color change (e.g., colorimetric), a fluorescent signal (e.g., using a portable UV light), a phosphorescent signal (e.g., after exposure to natural light), and a polymerization of a mixture upon contact with the suspected target molecule. The difference of the approaches described herein is the suspected target molecule is used a catalyst in the detection reaction. As such the suspected target molecule is not consumed during the course of the reaction and will continue to facilitate the completion of the reaction used for detection of the presence of the suspected target molecule. As a catalyst in the reaction, detection of the suspected target molecule is orders of magnitude more sensitive than approaches of detecting the suspected target molecule where there is a need for stoichiometric consumption of the analyte. The presence of a suspected target molecule that is utilized as a catalyst, and not as a reactant, allows a reaction to be initiated by ultra-low quantities of the suspected target molecule, if present, thereby highlighting the innate sensitivity of the described detection approaches. Likewise, the use of excess, stoichiometric amounts, etc. of the suspected target molecule will also produce a signal.

The mode of action of morphine, heroin, and opioid derivatives such as hydrocodone, codeine, oxycodone, etc. and including synthetic derivatives such as fentanyl, involves binding of the opioid molecule to the u-opioid receptor (MOR) in the brain. Activation of the MOR leads to the signature analgesic effects of the opioid as well as sedation, euphoria, and respiratory depression (as well as other physiological effects).

The catalytic nature of opioids and opioid derivatives as illustrated in FIG. 2A arises from the tertiary nitrogen (e.g., tertiary amine, 3° amine) in the tetracyclic core of the opioid molecular structure. Opioid molecules having similar structure with a tertiary amine in the tetracyclic core include heroin, hydrocodone, codeine, oxycodone, etc.

Heroin is an acetylated version of morphine which enables the heroin molecule to efficiently penetrate the blood-brain barrier (BBB) once it is injected in the bloodstream (FIG. 2A). Heroin's highly efficient ability to cross the BBB is due to its high level of hydrophobicity conferred by the acetyl groups blocking the hydroxyl groups. Once across the BBB, the heroin molecule is deacetylated by non-specific esterases, rendering the heroin molecule to be a candidate for the u-opioid receptor (MOR) in the brain and thereby affecting the central nervous system (CNS) by inducing analgesic effects as well as euphoric effects that eventually develop into dependency and addiction upon repeated use.

The catalytic nature of synthetic opioids, such as fentanyl and fentanyl derivatives as illustrated in part (a) of FIG. 2B, arises from the piperidine ring that contains a protonatable tertiary nitrogen (3° amine) that is important for maximizing the opioid's binding affinity to its biological target. Part (b) illustrates two different examples (i) and (ii) of the binding of the fentanyl molecule to the binding site of the u-opioid receptor (MOR), specifically the piperidine ring containing the tertiary nitrogen (as indicated by a dashed circle) in the binding site of the receptor interacting with carboxylate groups within the receptor. This tertiary nitrogen is protonated at physiological pH and can interact with both negatively charged carboxylate groups from aspartic acid residue (D147) or with the imidazole ring of the nearby histidine (H297). Chemical modification of this tertiary nitrogen, via oxidation (e.g., to give the N-oxide) or alkyl group side chain removal to generate norfentanyl renders the opioid inactive. Current detection methods of these opioid and opioid derivatives depend on stoichiometric or excess amounts of the suspected target drug to participate as a reactant in the detection methods.

Tropane alkaloids are bicyclic alkaloids having a pyrrolidine and a piperidine ring share a common nitrogen atom in their chemical structure. Moreover, for the compounds illustrated in FIG. 2C, atropine, cocaine, and scopolamine, the common nitrogen atom shared by the pyrrolidine and piperidine rings is tertiary nitrogen atom is an important component of the molecule for binding to the molecule's respective receptor in the human body. For instance, the tertiary amine in the atropine molecule is important for binding to the muscarinic acetylcholine receptor. In the case of the scopolamine molecule, the tertiary amine in the bicyclic ring system is responsible for its biological activity when the nitrogen is protonated at physiological pH. In addition, the molecule has two reactive functionalities: a unique epoxide unit is present in the bicyclic core and a primary alcohol unit present in the side chain. The last two structural motifs are chemical handles that can be exploited for the modification of the drug. Current detection methods of these tropane alkaloid type drugs depend on stoichiometric or excess amounts of the suspected target drug to participate as a reactant in the detection methods.

As illustrated in FIG. 2D, indole-based drugs, such as LSD and 1P-LSD that has an amide moiety on the nitrogen of the indole ring system, have a tertiary nitrogen in the molecule's tetracyclic core that is important for binding of the LSD and IP-LSD molecules to their respective biological receptor. Current detection methods for the LSD molecule, e.g., Ehrlich's reagent, does not work for IP-LSD because the nitrogen in the indole ring system is blocked by the amide moiety. Moreover, current methods of detection of the molecules depend on stoichiometric or excess amounts of the suspected target drug to participate as a reactant in the detection methods.

For each of the potentially suspected target molecules described herein, the tertiary nitrogen of the molecule is important for binding the molecule to its biological receptor. Moreover, each of these suspected target molecules can act as a catalyst in specific reactions. The catalytic nature of these suspected target molecules arises from the tertiary nitrogen, e.g., the tertiary nitrogen in their tetracyclic core of opioids and indole-based drugs, the tertiary nitrogen in the piperidine ring of fentanyl and analogs of fentanyl, the tertiary nitrogen in the bicyclic ring system of tropane alkaloids, etc. According to one embodiment, the catalytic feature of the indole-based drugs, i.e., the tertiary nitrogen in its tetracyclic core, is used to detect the presence of the molecule on a surface, in a product, etc. According to one embodiment, for detection of these suspected target molecules, the tertiary nitrogen moiety of each target molecule is used as a catalytic entity to bring about reactions between a number of reactants that alone or without the presence of the target molecule will not react at ambient temperature.

According to various embodiments, detection methods are described that use the structural feature of the tertiary nitrogen of the suspected target molecule to detect the presence of the molecule. The tertiary nitrogen moiety of the suspected target molecules may be used as a catalytic entity to initiate and complete reactions between a number of reactants that alone or without the presence of the catalyst will not react at ambient temperature.

As described herein, various embodiments include method of detecting suspected target molecules in any environment and at the site of exposure via a signaling event that can be a color change (e.g., colorimetric), fluorescent signal (e.g., using a portable UV light), phosphorescent signal (e.g., after exposure to natural light) and the polymerization of a mixture upon contact with the suspected target molecule. These methods are distinct from conventional drug detection methods because the suspected target molecule is used as a catalyst and not used as a reactant. As such, the suspected target molecule is not consumed during the course of the reaction and will continue to facilitate its completion. Moreover, the use of the suspected target molecule to function as a catalyst (and not as a reactant) allows initiation of a detection reaction in the presence of ultra-low quantities of the catalyst and thereby highlights the innate sensitivity of this detection approach. Likewise, the use of excess or stoichiometric amounts of the suspected target molecule will also produce a signal.

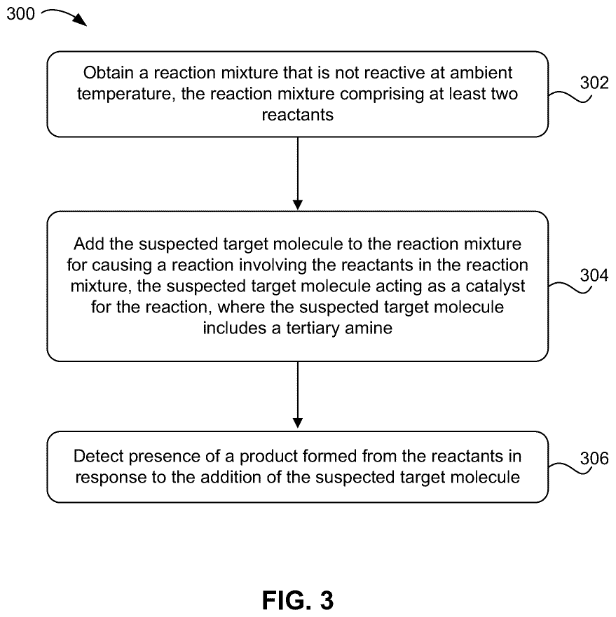

FIG. 3 shows a method 300 for detecting a suspected target molecule by using the suspected target molecule as a catalyst for a reaction, in accordance with one embodiment. As an option, the present method 300 may be implemented to construct structures such as those shown in the other FIGS. described herein. Of course, however, this method 300 and others presented herein may be used to form structures for a wide variety of devices and/or purposes which may or may not be related to the illustrative embodiments listed herein. Further, the methods presented herein may be carried out in any desired environment. Moreover, greater or fewer operations than those shown in FIG. 3 may be included in method 300, according to various embodiments. It should also be noted that any of the aforementioned features may be used in any of the embodiments described in accordance with the various methods.

According to one embodiment, the method 300 begins with operation 302 including obtaining a reaction mixture that is not reactive at ambient temperature, the reaction mixture comprising at least two reactants. The reaction mixture is comprised of reactants that utilize a molecule having a tertiary amine as a catalyst to enable the reactants to react with each other. In some approaches, the reaction mixture may include an additive. The additive may include a carrier solution, a solvent, a dye, etc. In some approaches, the solvent may be selected to enhance the color change resulting from the formation of a product between the reactants.

In some approaches, the reaction mixture may include more than two reactants. In one approach, the reaction mixture may include three reactants whose reactivity is catalyzed by a tertiary amine catalyst thereby forming a product in the presence of the tertiary amine catalyst. For example, a reaction mixture may include a first reactant being a thiol, a second reactant being an isocyanate, and a third reactant being an alcohol.

Operation 304 includes adding the suspected target molecule to the reaction mixture for causing a reaction involving reactants in the reaction mixture. The suspected target molecule acts as a catalyst for the reaction. Preferably the reactants react with each other in the presence of the catalyst. The temperature of the reaction is at ambient temperature (less than 40° C., e.g., room temperature). The temperature is below a spontaneous reaction temperature of the reaction mixture. The reactants are shelf-stable at ambient temperature. In some approaches, during a detection process, the reactants may be heated or cooled.

The suspected target molecule includes a tertiary amine. A tertiary amine is a nitrogen atom that has three alkyl groups attached to the nitrogen atom. The three alkyl groups are essential in the definition of the tertiary amine, such that if any one of the groups attached to the nitrogen atom is not an alkyl group and instead is an acetyl group, a carbonyl group, etc., the molecule cannot function as a catalyst. The presence of a tertiary amine in the suspected target molecule allows the suspected target molecule to function as a catalyst in specific reactions. The molecule having a tertiary amine does not react with the reactants of the reaction mixture, but rather initiates reactivity between the reactants to generate a product specific to a chemical reaction of the reactants. The reaction is chemoselective for a tertiary amine catalyst such that a molecule having a secondary or primary amine cannot function as a catalyst for the reaction. Rather, a molecule having a secondary or primary amine will react with the first reactant, second reactant, etc. only and generate a product formed from the reaction of the molecule having a secondary or primary amine and the reactant. A primary and/or secondary amine reacts with the reactants in a non-catalytic manner.

In various approaches, an amount of tertiary amine catalyst added to the reaction mixture may be less than 0.5 mol. % of the reaction mixture. In some applications, a trace amount of a tertiary amine catalyst may be sufficient to act cause the reaction to generate a product. In some approaches, a tertiary amine catalyst may be present in a very small amounts, such as 1-10 μg, and may be detected with a described reaction system. An amount of tertiary amine catalyst may be present in excess amounts without adversely affecting the reactivity of the reactant, e.g., causing a diminished amount of product generated. The suspected target molecules having a tertiary amine are typically in the form of a solid (i.e., acid form) or an oil (i.e., free-base form).

Moreover, the molecule having a tertiary amine cannot have a reactive secondary amine or a reactive primary amine in the same molecular structure. The presence of a reactive secondary amine or reactive primary amine may eclipse the presence of the tertiary amine thereby causing the secondary amine or primary amine to react with one of the reactants in the reaction mix (even though a tertiary amine is present to catalyze a reaction between the reactants). Furthermore, a tertiary amine having an aniline core does not act as a catalyst for the reactions described herein. In one example, fentanyl includes a tertiary amine and a nitrogen in an amide group (see part (a) of FIG. 2B), however, the amide group is non-basic and will not be involved in the reaction with reactants.

Although it is preferably that primary and/or secondary amines are not present with the suspected target molecule, it is unlikely that primary and/or secondary amines would be present in an environment with the suspected target molecules because amines have a strong scent (i.e., an ammonia smell), and liquid amines have a distinctive fishy and foul smell.

Generally available molecules having a tertiary amine that act as a catalyst in the described reactions include triethylamine which is a common base used in organic synthesis. However, a difference of this molecule compared to the suspected target molecule described herein is triethylamine is a small molecule and exists as a liquid. Another tertiary amine catalyst is Hunig's base (diisopropylethylamine) is another common base that also exists as a liquid. These liquid tertiary amines also have a distinctive ammonia smell and, thus, would not be found with narcotic drugs. These tertiary amines are typically found only in a chemistry laboratory, obtained from a chemical manufacturer, such that these tertiary amines are not commonly found in any natural environment.

The reaction mixture described herein uses a tertiary amine catalyst as a conduit between the reactants for forming a product. The tertiary amine catalyst brings the reactant molecules together so that the reactants react with each other, and the reactant do not react with the tertiary amine. The tertiary amine catalyst does not react with any of the reactants to form an intermediate.

In some reaction mixtures, a tertiary amine may act as a catalyst by forming an intermediate with one of the reactants so that the second reactant interacts with the intermediate to form a product without the tertiary amine. These types of reaction mixtures are not optimal for the applications described herein. The activation energy for the first reaction of the tertiary amine with reactant B needs to be sufficiently low for the intermediate to be formed at ambient temperature. For example, the complexity of the tertiary amines in the suspected target molecules described herein may not be efficient catalysts for forming the intermediates essential for the reaction between two reactants. For example, the Bayliss-Hillman reaction utilizes a tertiary amine as a catalyst for the reaction between an activated alkene with an aldehyde. Typically, the tertiary amine catalyst for the Bayliss-Hillman reaction may be an unhindered tertiary amine such as DABCO (1, 4-diazabicyclo[2.2.2.]octane). However, a reaction mixture of an activated alkene and an aldehyde in the presence of a suspected target molecule having a tertiary amine, e.g., fentanyl, there is no reactivity between the reactants. Without wishing to be bound by any theory, it is believed that the steric hindrance near the tertiary amine in the fentanyl molecule, or any of the suspected target molecules described herein, causes the activation energy of the associate of the tertiary amine with the initial reactant to be so high that the intermediate between the tertiary amine and the initial reactant does not form and the reaction does not proceed to form a product with the second reactant.

In some approaches, a suspected target molecule is an opioid molecule, where the opioid molecule has a tertiary amine. The opioid molecule may include morphine, heroin, hydrocodone, codeine, oxycodone, etc. as illustrated in FIG. 2A. In one approach, the opioid molecule is a synthetic opioid such as fentanyl, analogs of fentanyl, etc. as illustrated in FIG. 2B.

In some approaches, a suspected target molecule is an alkaloid, such as a tropane alkaloid, where the tropane alkaloid molecule has a tertiary amine. The tropane alkaloid may include atropine, cocaine, scopolamine, etc. as illustrated in FIG. 2C.

In some approaches, a suspected target molecule is an indole-based molecule, where the indole-based molecule has a tertiary amine. The indole-based molecule may include LSD, IP-LSD, derivatives of LSD, etc. as illustrated in FIG. 2D.

Referring to FIG. 3, operation 306 includes detecting presence of a product formed from the reactants in response to the addition of the suspected target molecule, where the suspected target molecule functions as a catalyst for the reaction. A change in the product may indicate any of the suspected target molecules described herein, and further, may indicate suspected target molecules that have a tertiary amine not described herein. The method provides an immediate indication of the presence of a suspicious target molecule. Further methods of identification will be used to identify the type of target molecule present that has been detected. Such methods may include hand held mass-spectrometers such as a Raman spectrometer, a portable GC-MS instrument, FTIR spectroscopy, lateral flow assays, test strips, etc. In particular, portable GC-MS instruments offer an analytical technique for site evaluation (e.g., housed in a van or another mode of transportation). Portable GC-MS instruments are highly versatile and contain internal libraries that can provide a quick identification of the material at the collection site.

### Reaction Between Thiols and Isocyanates

In one embodiment, the reaction mixture includes a thiol reactant and an isothiocyanate reactant. The reaction between thiols and isocyanates does not occur when these are mixed in a reaction vessel at ambient temperature. The reaction between thiols and isocyanates does not occur below about 60° C. Without the addition of any other reagents, the reaction between thiol and isocyanate occurs at elevated temperatures with varying yields of product (>60° C., 60-81% yields). Including a tertiary amine in the reaction mixture may allow the tertiary amine (e.g., a molecule having a tertiary nitrogen) to act as a useful and efficient catalyst for the reaction between a thiol (SH) group and an isocyanate (N═C═O) at ambient temperature (25° C.). Furthermore, some tertiary amines, such as tertiary amines having an aniline core do not function as a catalyst in this reaction, and thus, including a tertiary amine having an aniline core in a reaction mixture of a thiol and an isocyanate would not catalyze the reaction.

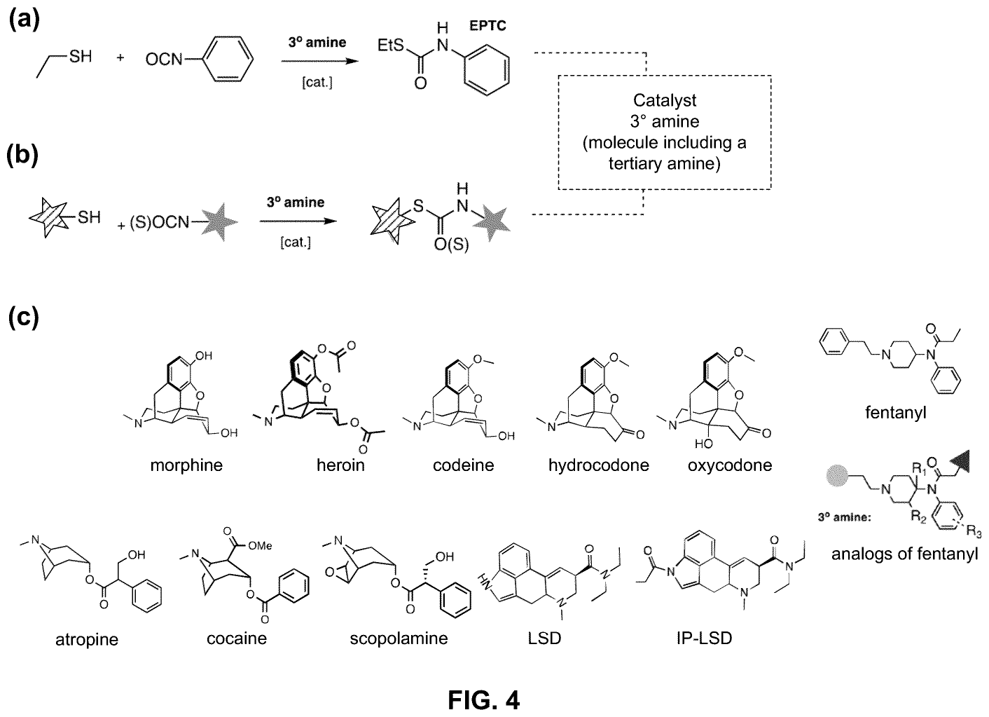

As illustrated in the schematic drawing of FIG. 4, in one example, in part (a) a reaction mixture of ethanethiol and phenylisocyanate generates a thiocarbamate (e.g., EPTC) in the presence of a tertiary amine catalyst. At room temperature, the reaction between the reactants ethanethiol and phenylisocyanate only yields EPTC in the presence of a tertiary amine, a tertiary amine being a molecule containing a tertiary nitrogen. For instance, as illustrated in part (c), tertiary amine may be an opioid or opioid derivative, a tropane alkaloid such as atropine, cocaine, and scopolamine, LSD, LSD derivatives, etc. The union of the thiol and the isocyanate generates an S-thiocarbamate product as illustrated in FIG. 4. As illustrated in part (b), the schematic drawing represents a generalized form of the reaction between a thiol and an isocyanate, where the striped star and solid start represent a non-participating, reporting moieties (e.g., a dye).

The colorimetric nature of the reaction may be anticipated if both reaction partners (reactants thiol and isocyanate) react to produce a colored solution. Early approaches of combining ethanethiol and phenylisocyanate in nitromethane in the presence of a molecule having a tertiary nitrogen as listed in part (c) has yielded a colored solution (e.g., yellow) within 1-2 minutes that progressively brightens over time, the final color of the solution being bright orange after 1 hour at ambient temperature.

Several important aspects of this reaction make it an attractive platform for devising methods for the detection of molecules having a tertiary amine moiety illustrated in part (c). A first aspect is the reaction between the thiol and the isocyanate occurs, albeit poor yields, only at elevated temperatures (>60° C.) and the reaction does not occur at ambient temperature. A second aspect of the reaction is the reaction may occur rapidly and efficiently at room temperature only in the presence of a tertiary amine catalyst.

A third aspect is the reaction is chemoselective. Chemoselective is defined as the reaction can only be catalyzed by a tertiary amine, such that primary and secondary amines, as well as alcohols and amides do not catalyze the reaction. The chemical reaction between a thiol and an isocyanate is a highly selective platform for molecules exhibiting a tertiary amine moiety.

A fourth aspect of the reaction is that the tertiary amine, by virtue of being a catalyst, may be used in concentrations down to 5 mol. % relative to each reactant, e.g., the thiol or the isocyanate component. In reactions of polymerization of the reactants, concentrations of the tertiary amine catalyst may be less than 5 mol. % relative to each reactant.

Based on the aspects described above, a reaction mixture of a thiol and an isocyanate (part (b)) is an attractive platform to be used in detection methods for suspected target molecules, such as opioids, opioid derivatives, tropane alkaloids, LSD and LSD derivatives, etc. as illustrated in (c), FIG. 4. To this end, initial experiments evaluating a test system has produced successful results that open a myriad of potential applications in biochemistry, materials science and forensic chemistry. Due to the nature of the reaction, isothiocyanates would behave similarly, thus opening up a set of other substrates that can be explored for devising detection methods for suspected target molecules having a tertiary amine, such as those listed in part (c).

In one approach, a reaction mixture includes thiol reactant that include a donor dye and an isothiocyanate reactant that includes an acceptor dye. Part (b) illustrates a generalized form of a reaction where the thiol reactant is a donor dye having a non-participating, reporting moiety (stripe patterned star) and the isocyanate reactant, e.g., an isothiocyanate reactant, is an acceptor dye having a non-participating, reporting moiety (solid star). The product includes a predefined fluorescent signal resulting from the reaction of the thiol reactant and the isocyanate reactant in the presence of the suspected target molecule having a tertiary amine. In one approach, the isothiocyanate reactant is a fluorescein isothiocyanate. For some fluorescence applications, the activation of a fluorescent dye may occur by means of Förster resonance energy transfer (FRET).

### Reaction Between Nitroalkanes and Isocyanates

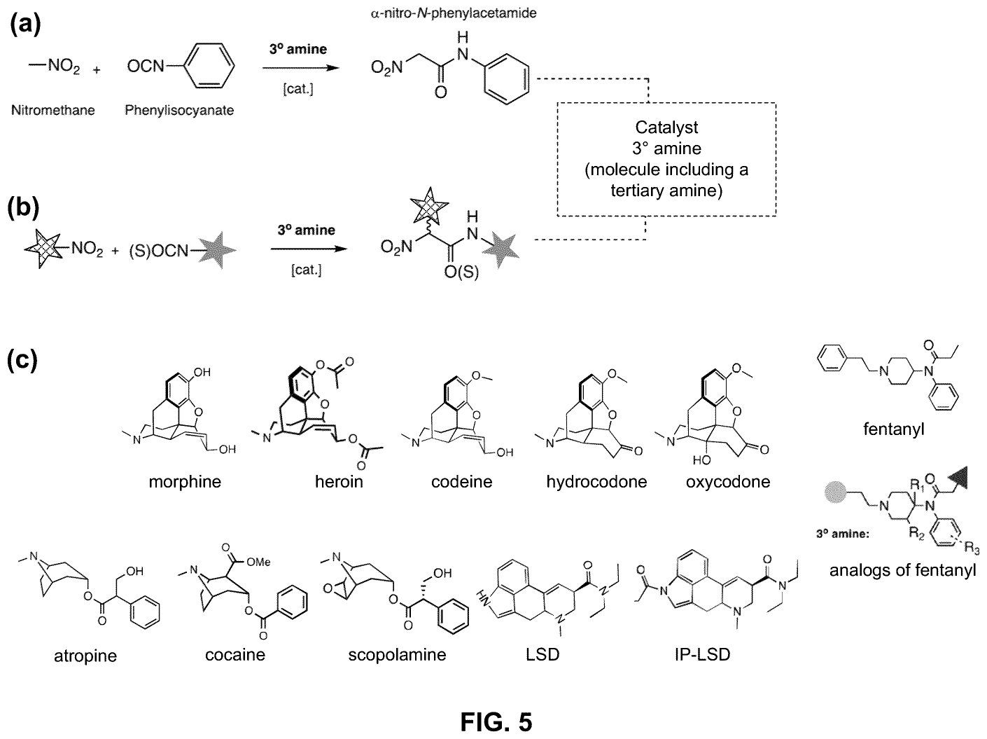

In one embodiment, the reaction mixture includes a nitroalkane reactant and an isocyanate reactant. The reaction may be catalyzed by a molecule having a tertiary amine to result in a product. The reaction between a nitroalkane reactant and an isocyanate occurs at ambient temperature in the presence of a catalyst tertiary amine. As illustrated in the schematic drawing of part (a), FIG. 5, at ambient temperature, a reaction mixture of nitromethane and a phenylisocyanate in the presence of a tertiary amine catalyst generates the product a-nitro-N-phenylacetamide. The reaction only yields a-nitro-N-phenylacetamide at room temperature in the presence of a catalyst, and the catalyst being a molecule with a tertiary amine such as opioid and opioid derivatives, tropane alkaloids, LSD and LSD derivatives, etc. as illustrated in part (c).

The reaction between nitromethane and phenylisocyanate when carried out in a 1:1 stoichiometric ratio of these reactants in the presence of a tertiary amine catalyst, generates α-nitro-N-phenylacetamide as one of the main products (˜60% by EI-GC-MS analysis). In the reaction mixture, if other competing nucleophiles are present in excess, such as thiols, alcohols, etc. then products such as S-thiocarbamates, O-carbamates, respectively, may be generated and may be present as the dominant product in the product distribution.

In one approach, a specific application of a suspected target molecule having a tertiary amine as listed in part (c), for example, in the reaction of a nitroalkane and an isothiocyanate to generate the formation of a-nitro-N-phenylacetamide may be expanded for the use of other starting partners bearing different probes that can be joined together or brought into close proximity to cause a signaling event. As it can be expected, the use of any analog of these molecules having a tertiary amine as listed in part (c) may act as a catalyst in the reaction to a similar extent in catalyzing the reaction as the molecular structure of the suspected target molecules contain a tertiary amine.

Part (b) illustrates a generalized form of a reaction where the nitroalkane reactant is a donor dye having a non-participating, reporting moiety (cross-hatched star) and the isocyanate reactant, e.g., an isothiocyanate reactant, is an acceptor dye having a non-participating, reporting moiety (solid star). The product includes a predefined fluorescent signal resulting from the reaction of the nitroalkane reactant and the isocyanate reactant in the presence of the suspected target molecule having a tertiary amine. In one approach, the isothiocyanate reactant is a fluorescein isothiocyanate. For some fluorescence applications, the activation of a fluorescent dye may occur by means of FRET technology.

### Reaction Between Alcohols and Isocyanates

In similar fashion to the reaction between thiols and isocyanates, alcohols are a class of compounds that also reacts with isocyanates in the presence of tertiary amine catalysts. In one embodiment, a reaction mixture includes an alcohol reactant and an isocyanate reactant. For example, alcohols and isocyanates participate in polymerization reactions that may be used as an application of detection. The proposed mechanism resembles the one by which the amine catalyzes the reaction between thiols and the isocyanate counterpart. As the amine basically acts as a conduit to bring together both reactive species via hydrogen bonding and gets released once the formation of the carbamate is realized, its role is solely catalytic. Again, the fact that the tertiary amine as a catalyst is important as it means that very small amounts of it (1-10 μg) may be detected with a proper reaction system.

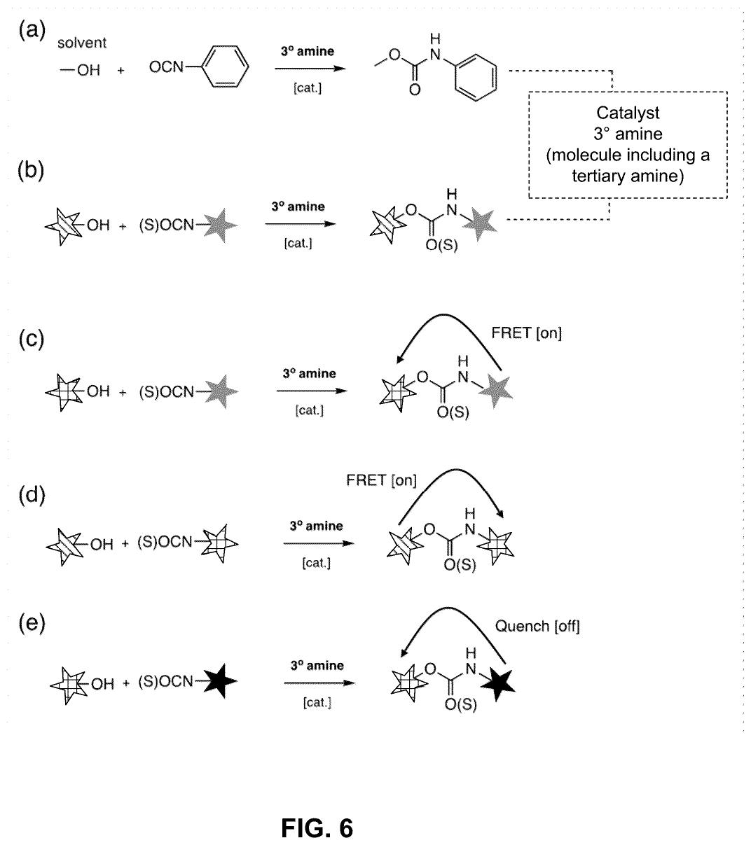

As illustrated in FIG. 6, colorimetric applications using this particular reaction can be realized if we combine dyes bearing available hydroxyl groups for reaction with a given isocyanate. Two forms of color change may be accomplished in this manner, one by virtue of expanding the conjugation of a given dye upon formation of a carbamate linker. As illustrated in part (a), a reaction between methanol and phenylisocyanate generates a carbamate product in the presence of a tertiary amine catalyst (e.g., an opioid, an opioid derivative, a tropane alkaloid, LSD, LSD derivative, etc.). Reaction only yields product (detected by EI-GC-MS) at room temperature in the presence of the tertiary amine.

Part (b) illustrates a generalized form of a reaction where the alcohol reactant is a donor dye having a non-participating, reporting moiety (striped pattern star) and the isocyanate reactant, e.g., an isothiocyanate reactant, is an acceptor dye having a non-participating, reporting moiety (solid pattern star). The product includes a predefined fluorescent signal resulting from the reaction of the alcohol reactant and the isocyanate reactant in the presence of the suspected target molecule having a tertiary amine. For some fluorescence applications, the activation of a fluorescent dye may occur by means of FRET technology.

As illustrated in parts (c) and (d), a FRET approach includes the alcohol acceptor fluoresces only when the reaction has occurred bringing the donor dye (as the isocyanate) close in proximity. The FRET effect is indicated by the black arrow. Within the context of FRET, care must be taken in choosing the right donor: acceptor combinations for the approach to work effectively and provide an “ON” signaling event (parts (c) and (d)). It is noteworthy mentioning that the acceptor and donor roles can be fulfilled by either component of the reaction.

Therefore, the fluorescence acceptor in one case can be the alcohol as depicted by the grid pattern star in part (c) while it can be the isocyanate a depicted by the grid pattern star in the example of part (d). Lastly, one may envision a fluorescence “OFF” event where a quencher may be brought together using this chemistry and due to its proximity now can quench the dye's fluorescent signal. As illustrated in part (e), therefore the fluorescent dye (shown here with the grid pattern star on the alcohol, but the fluorescent dye may be the isocyanate) is quenched by another dye as depicted by the solid black star (e.g., azo dye). This is a form of fluorescence OFF mechanism. Although fluorescence “OFF” approaches are seldom used and not very attractive from a signaling perspective, they can still be of use in certain scenarios if fluorescence “ON” approaches are difficult to implement. Again, the role of the acceptor can be fulfilled by the species bearing the alcohol or the isocyanate. As stated above, the use of isothiocyanates in place of the isocyanates here can be expected as a similar reaction pathway would be at work. Each of these proposed reactions only proceed at ambient temperatures in the presence of a tertiary amine catalyst such as the suspected target molecules described herein.

According to various approaches, the presence of a suspected target molecule may be detected by a color change in the reaction mixture that is caused by the formation of the product. The product formed by the chemical reaction of two reactants in the presence of a tertiary amine catalyst causes a color change in the reaction mixture and, thus, the tertiary amine catalyst being a suspected target molecule may be detected based on the color change. In one approach, the color change may be colorimetric change in color in the reaction mixture. In another approach, the color change may be a change in fluorescent color in the reaction mixture.

### Involvement in Polymerization Reactions

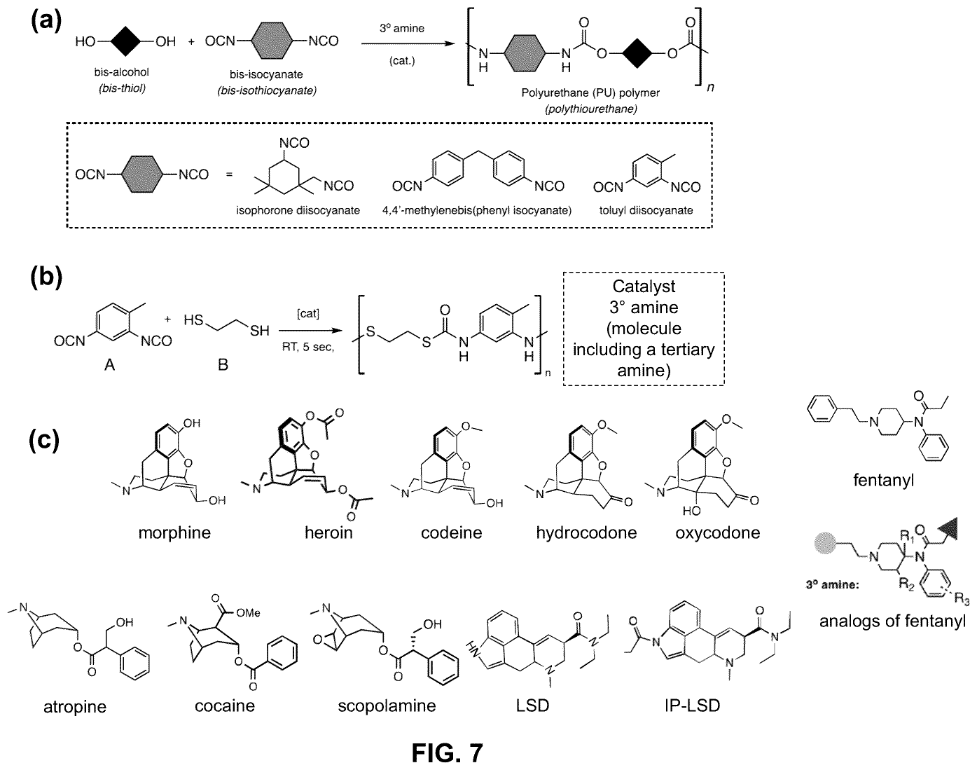

Based on specific results obtained for the thiol-isocyanate reaction, a particular application that was found interesting was in polyurethane synthesis. In one embodiment, a reaction of at least two reactants includes a polymerization reaction initiated by the presence of a suspected target molecule, where the product is a polymer. In one approach, the product formed from the polymerization reaction is a polyurethane. As illustrated in part (a) of FIG. 7, for example, polyurethanes are a class of polymer that is constructed by the union of a bis-alcohol (e.g., ethylene glycol) and a bis-isocyanate (4,4′-methylenebis(phenyl isocyanate)). This polymerization reaction may be catalyzed by a variety of tertiary amines in industry and as such our initial proposal is that different initial building blocks, toluyl diisocyanate (A) and 1,2-ethanedithiol (B), as illustrated in part (b), can be engaged in a polymerization reaction with each other in the presence of catalytic amounts of a catalyst, such as the tertiary amine catalysts illustrated in part (c).

An interesting scenario that can occur during the deployment of this technology in the field is that even if a white solid that looks like a suspected target molecule (e.g., any one of the suspected target molecules of part (c)), for example an inorganic salt like sodium carbonate, although can still catalyze the reaction, it will have to be soluble in dichloromethane or another organic solvent prior its introduction to the dithiol:diisocyanate mixture. Therefore, by taking the solid up in an organic solvent prior to its introduction to the dithiol:diisocyanate mixture and watching whether or not dissolves, one can discard the solid as a potential synthetic opioid even if this one is its salt form.

### Production of Phosphorescence-Yielding Adducts

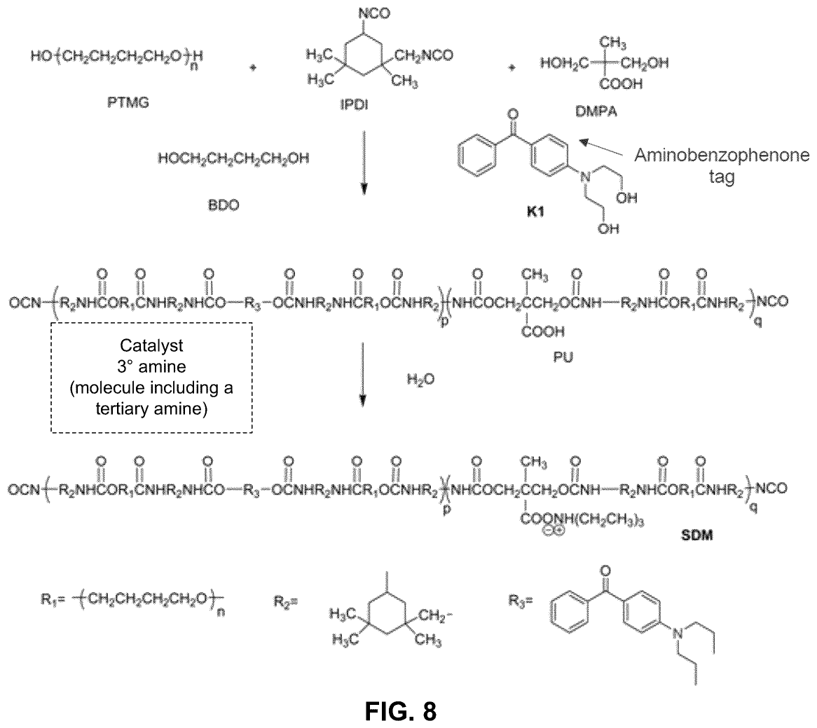

In one approach, formation of phosphorescent polymer may indicate the presence of a suspected target molecule. For example, a suspected target molecule may be used as a catalyst in the production of phosphorescent polyurethanes. Building blocks used to produce this phosphorescent material may utilize a molecule having a tertiary amine as a catalyst in the polymerization reaction. In one approach, a polymerization reaction includes an aminobenzophenone derived polyurethane polymer. As illustrated in FIG. 8, a polymerization reaction may include fluorescent and phosphorescent PU synthesis approach. The role of the aminobenzophenone unit is to act as a fluorescent tag but then a phosphorescent tag upon polymer formation. The role of the catalyst having a tertiary amine such as the suspected target molecules described herein will act as a catalyst to initiate and complete the polymerization and convert it into a phosphorescent polymer.

### Direct Fluorescent Detection

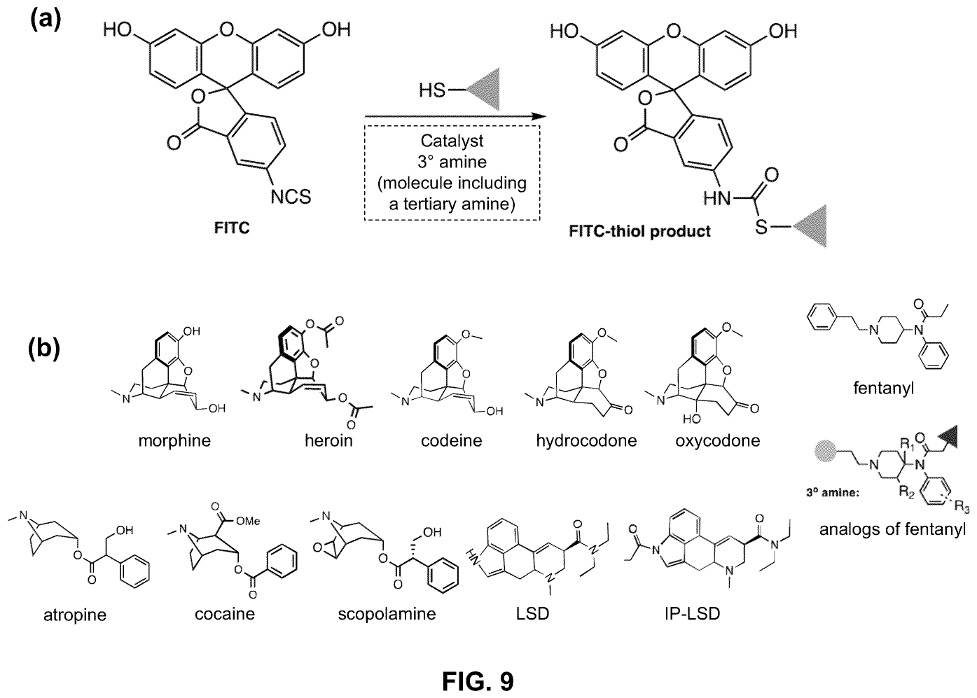

In another approach, a fluorescence detection system may involve the use of a fluorescent dye called fluorescein isothiocyanate (FITC). The reaction between ethane thiol and FITC may occur in the presence of a suspected target molecule (e.g., atropine). As illustrated in part (a) of FIG. 9, a proposed reaction between the dye FITC (fluorescein isothiocyanate) and a thiol-bearing molecule will run in the presence of a catalyst having a tertiary amine. Examples of tertiary amine catalysts that may be present at suspected target molecules are shown in part (b).

### Direct Fluorescent Detection Using a Modified Hinsberg Reaction

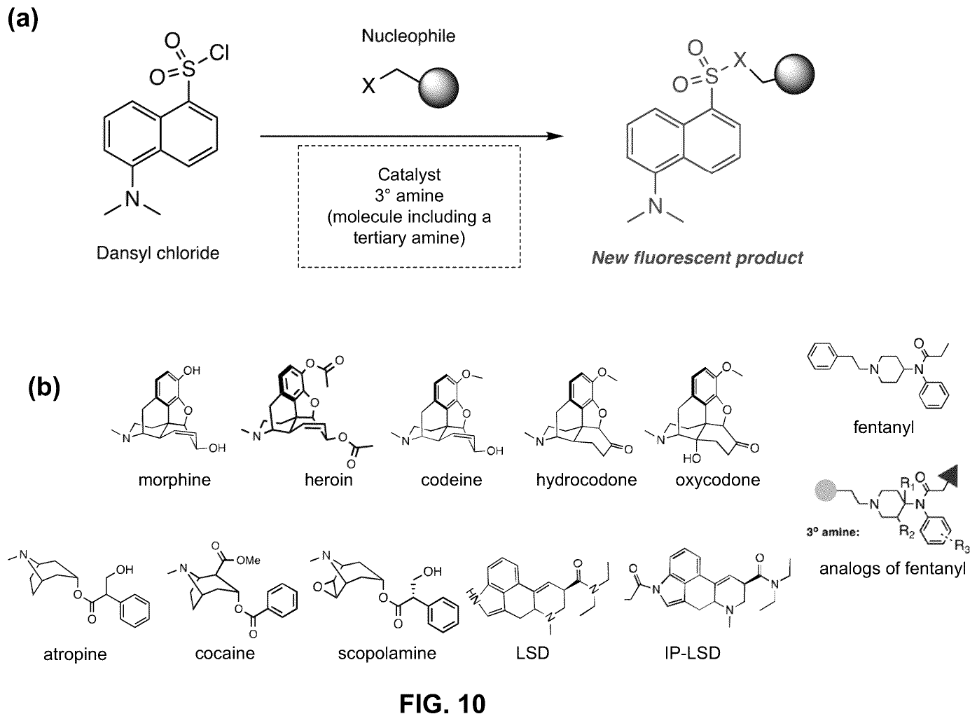

A reaction between a sulfonyl chloride (e.g., dansyl chloride), a fluorogenic sulfonyl chloride dye, and a nucleophile in the presence of catalytic amounts of a molecule having a tertiary amine yields a product that is fluorescent in nature. As illustrated in part (a) of FIG. 10, a starting dansyl chloride dye may be fluorescent at the start of the reaction, but this fluorescent emission may change by a reaction with a suitable nucleophile (e.g., alcohol, amine, thiol, etc.) in the presence of a molecule containing a tertiary amine. Examples of tertiary amine catalysts that may be present at suspected target molecules are shown in part (b).

The sulfonyl chloride partner may be any sulfonyl chloride-based dye. This is a modification of the Hinsberg test that states that tertiary amines do not lead to the formation of any products. The target molecule containing a tertiary amine acts as a catalytic entity facilitating the union of a nucleophile with the sulfonyl chloride. In one example, a suspected target molecule (e.g., any one of the molecules shown in part (b)) acts as a catalyst for the reaction between dansyl chloride and an ethanol solvent that would yield a fluorescent product when irradiated by a handheld, portable UV-lamp (λ=365 nm). Other solvents that may be used include: methanol, ethylene glycol, isopropanol, etc.).

### Applications for Detection of a Suspected Target Molecule

According to various approaches, the highly specific chemoselectivity of the reaction (i.e., tertiary amines), different applications of the reaction may provide initial platforms for further investigations. Regarding the high selectivity for tertiary amines, there is a characteristic of these species that must be identified. Most tertiary amines found in the laboratory setting are liquids are room temperature (e.g., triethylamine, diisopropylethylamine, etc.) while various suspected target molecules having a tertiary amine as described herein (see FIGS. 2A-2D) even in its base form are solids (off-white, cream-colored solids). Therefore, this additional piece of information helps to narrow down the possibilities of a given white→off-white solid being a material other than an opioid.

### Magic Spray

In one embodiment, a solution for detecting a suspected target molecule includes a reaction mixture that includes at least two reactants and a solvent. The reaction mixture, as described herein, is not reactive in the solvent at ambient temperature. The reaction mixture is configured to be reactive upon exposure to a suspected target molecule that acts as a catalyst, thereby causing the reaction mixture to react and form a product. The solution may be sprayed (e.g., a spraying solution) from a spray container onto a surface having the suspected target molecule. For example, the solvent may function as a carrier solution for the reaction mixture to be sprayed onto the suspected target molecule. In one approach, the solution may include only the reaction mixture and a solvent. In another approach, the solution may include an additive such as carriers, solvents, etc. An additive may be included as a component to enhance the spraying of the reaction mixture on a surface. The solution may include a combination of additives.

In one approach, for example, a spraying solution may include a 1:1 mixture of the thiol and the isocyanate in a carrier liquid that can be sprayed onto various surfaces presumed to be contaminated with a suspected target molecule (for example, at least one of the suspected target molecules described in FIGS. 2A-2D). Upon contact of the spraying solution with the suspected target molecule, the reaction between the thiol and the isocyanate is catalyzed by the suspected target molecule thereby leading to a color change. This color change may be visualized with a UV light (long and short wavelength), with the naked eye, etc. as the suspected target molecule interacts with the spraying solution. A spraying solution for detection is especially useful for visualizing contaminated surfaces where a suspected target molecule (e.g., at least one of the suspected target molecules described in FIGS. 2A-2D) may be present in trace amounts (circulating currency, cleaned table surfaces, doorknobs, etc.).

### TLC-Blotter Paper Strip Technology

The product of the reaction forms on the applied surface, e.g., surface of the substrate containing the suspected target molecule. The suspected target molecule may be present on the surface and/or present in the substrate. For example, the solution having the reaction mixture may penetrate a substrate having the suspected target molecule and the product formed form the reaction of the reactants in the presence of the suspected target molecule would cause a change in color inside the substrate. In one approach, a detection method may include applying the reaction mixture to blotter papers, where the blotter paper is spiked with a suspected target molecule (e.g., atropine, LSD, LSD derivatives, etc.). For example, in some cases, a blotter paper spiked with a suspected target molecule may be created for recreational use. In one approach, a blotter paper that contains the suspected target molecule in specific concentrations is treated with a reaction mixture of a thiol/isocyanate pair of reactants that would generate a color change upon interaction with suspected target molecule in the blotter paper.

### Histological Applications/Tissue Staining

In one approach, a reaction mixture having at least two reactants may be used as a staining mixture for biological or molecular biology applications. In one approach, the reaction mixture is applied to a substrate where the substrate includes the suspected target molecule (e.g., the substrate is a specimen). The staining mixture may be applied to the following samples: a biological specimen, a biological product, etc. The product of the reaction of the reactants in the presence of a suspected target molecule acting as tertiary amine catalyst causes a color change of portions of the sample, if the suspected target molecule is present in the sample.

For example, in one approach, technology described herein may be applied to interrogate collected biological tissues or live tissues for the presence or accumulation of a known target molecule in animal tissues when given in a precise dose in a precise way (e.g., at least one of the suspected target molecules described in FIGS. 2A-2D). This would allow researchers to better visualize the target molecule transported throughout the body (kinetics), and its accumulation in different tissue types. Again, a specific thiol/isocyanate pair may be employed. This application possesses great potential due to the highly selective nature of the reaction, with the likelihood of occurring even in the presence of tissue fixing agents.

In one approach, a scientist may employ an antibody that is conjugated to a dansyl chloride, FITC tag, etc. giving exquisite labelling of a specific cell type or cellular structure (vesicles) in a biological specimen (i.e., better resolution at the cellular level than flooding an entire biological specimen with a thiol/isocyanate pair). If the catalyst target molecule was also present in the area of the antibody binding to the cell or cellular structure, it could react per the chemistries described and give a fluorescence signal (with a shifted emission spectrum in the case of FITC vs the FITC thiol product).

### FRET-Based Fluorescence Approach

In one embodiment, the reaction described herein may be adapted for the fluorescence detection of suspected target molecules. One approach involves the use of FRET technology whereby a donor dye can be linked to a thiol-bearing unit that then react with an isocyanate unit that bears and acceptor dye. Due to the union of these two units containing what is known as a FRET-pair, one can envision the specific fluorescence signal arising from only a mixture where an opioid is present. Conversely, the donor dye can be linked to the isocyanate unit while the acceptor dye can reside with the thiol unit. FRET approaches are highly useful in biology and biochemistry as they provide specific fluorescence signals with little to no background fluorescence arising from either dye. This approach is presented in a more descriptive manner above. This approach includes a detection system that may include alcohols and thiols, as well as isocyanates and isothiocyanates.

### Substrate-Based Products for Detecting a Suspected Target Molecule

In one embodiment, a product for detecting a suspected target molecule includes a substrate and a reaction mixture coupled to the substrate. As described herein, the reaction mixture includes at least two reactants where the reaction mixture is not reactive at ambient temperature. The reactive mixture is configured to be reactive upon exposure to a suspected target molecule that acts as a catalyst thereby causing the reaction mixture to react and form a product. In some approaches, the reaction mixture is present on the surface of the substrate. In some approaches, the reaction mixture is present in the substrate.

### Developing Stick/TLC Plate

In one approach, the reaction mixture is present at a designated spot, stripe, etc. on a paper strip (e.g., a substrate) that is available as a test kit (e.g., similar to a pregnancy test kit). A sample of a given matrix material (e.g., urine, blood, sweat, saliva, etc.) may be applied to the bottom of the paper strip and as capillary action takes place, the matrix material interacts with the reaction mixture (e.g., a thiol/isocyanate reactant pair) at the designated spot, and upon interaction of a suspected target molecule in the matrix material with the reaction material, a product is formed on the paper strip, and detection of the product is possible using a signal as described herein, e.g., a colorimetric signal, a fluorometric signal, etc.

### Adhesive Patch Detection System

In one approach, a clear adhesive patch containing both reaction components when in contact is applied to a suspicious surface under examination, where upon exposure to a suspected target molecule, the previously clear adhesive patch will demonstrate a change in color, a polymerization, a fluorescence, etc. thereby indicating a tertiary amine catalyst is present causing the reaction to be initiated to form a product.

### Masks, Swabs, and HVA Filters

In some approaches, a substrate may be produced from electrospinning nanofibers. In one approach, the reaction mixture may be interspersed in the electrospun nanofibers. In one approach, the reactants of the reaction mixture may be incorporated into nanofiber non-woven materials such as custom face masks (e.g., N95, K95, etc.), swabs, HVAC filter materials, etc. For example, a mask including the reaction mixture may be worn in a high risk environment, and upon exposure to a suspected target molecule, the chemical reaction of the reactants of the reaction mixture is initiated in the presence of a tertiary amine (i.e., the suspected target molecule), and a product is formed in the mask as detected by a change in color thereby signaling an exposure hazard in the environment.

### Experiments

Reaction Between Thiol and Isocyanate with Atropine as a Catalyst

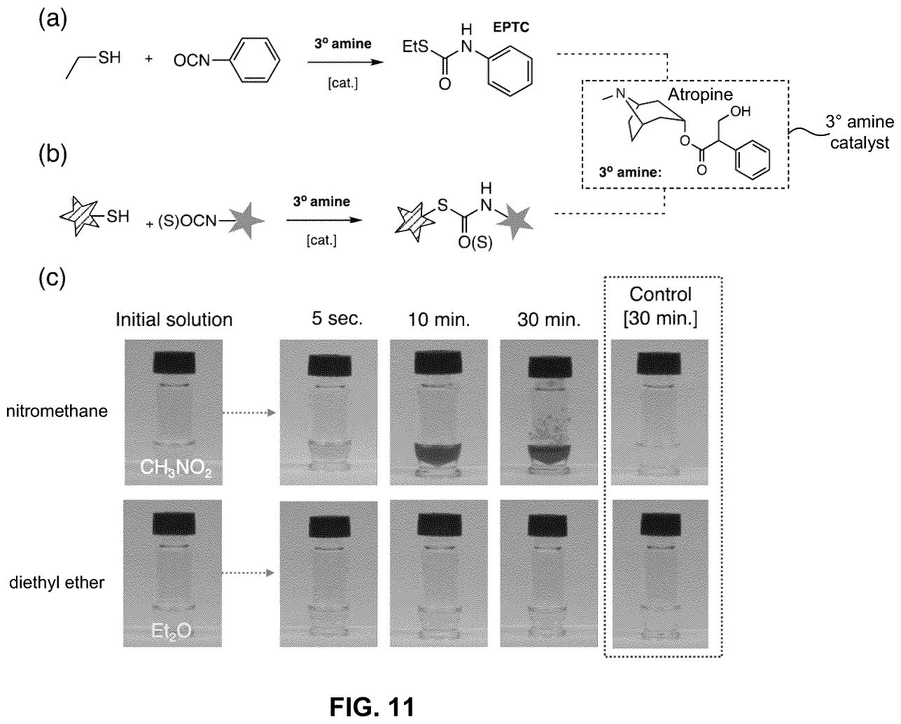

A colorimetric reaction was conducted using ethanethiol and phenylisocyanate in two different solvents, nitromethane and diethyl ether, in the presence of atropine as the catalyst. The schematic diagram of the reaction is illustrated in part (a) of FIG. 11. Part (b) illustrates a generalized form of a reaction, as described earlier in FIG. 4, where the thiol reactant may be a donor dye having a non-participating, reporting moiety (stripe patterned star) and the isocyanate reactant, e.g., an isothiocyanate reactant, may be an acceptor dye having a non-participating, reporting moiety (solid star).

The result of the reaction between ethanediol and phenylisocyanate in the presence of a catalyst may be demonstrated by a colorimetric test. As illustrated in part (c) of FIG. 11, the series of images represent the reaction between the reactants ethanediol and phenylisocyanate in a nitromethane [CH₃NO₂] solvent (upper panel) or diethyl ether [Et₂O] solvent (bottom panel) in the presence of a catalytic amount of atropine (˜5 mol. %). The arrow represents the point in time where the atropine is added in catalytic amount (˜5 mol. %) and the control vial contains all reaction components except atropine. There was a color change during the reaction in the nitromethane solution (upper panel), while no color change was observed during the reaction the diethyl ether solution (lower panel) as monitored for the duration of the reaction (t=30 min.). The product EPTC was formed in both the nitromethane solution and the diethyl ether solution as identified by Gas Chromatography-Mass Spectrometry analysis. The thiol component of the reaction in the diethyl ether reaction mixture caused a reactivity with the dye in the reaction such that the dye did not form a color when the EPTC was formed.

As time passed after addition of the atropine (dashed arrow), one can see that within 5 seconds, a yellow coloration was observed, and the yellow color increased in intensity and changed into a bright orange as time progresses (10 and 30 minutes). In contrast, the control experiment containing all reaction components except atropine remains colorless (Control at 30 minutes).

Reaction of Nitroalkane and Isothiocyanate with Suspected Target Molecule as Catalyst

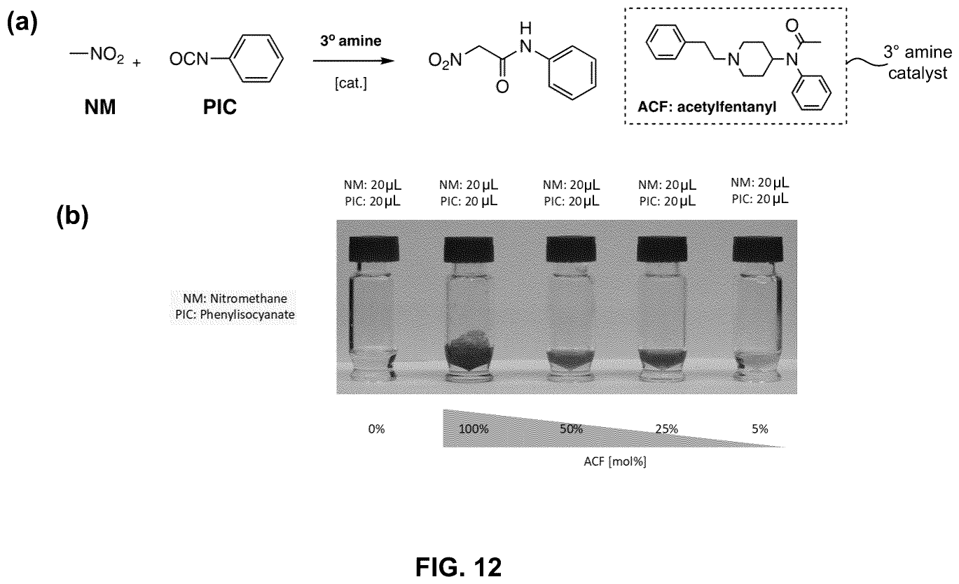

A reaction between nitromethane and phenylisocyanate occurs at ambient temperature with the aid of a tertiary amine catalyst, the catalyst being an acetylfentanyl molecule, part (a), FIG. 12.

The image of part (b) of FIG. 12 shows the results from a colorimetric reaction that may be used as a test for the detection of a suspected target molecule. A combination of equal stoichiometric amounts of nitromethane (NM) and phenylisocyanate (PIC) in the presence of various concentrations of acetylfentanyl (ACF) yielded a yellow solution within 1-2 minutes and the color progressively brightened over time. Again, due to the nature of the reaction, it is likely that isothiocyanates would behave similarly to isocyanates, thus opening up a set of other substrates that can be explored for devising detection methods for other suspected target molecules in addition to synthetic opioids.

Polymerization Reaction with Suspected Target Molecule as a Catalyst

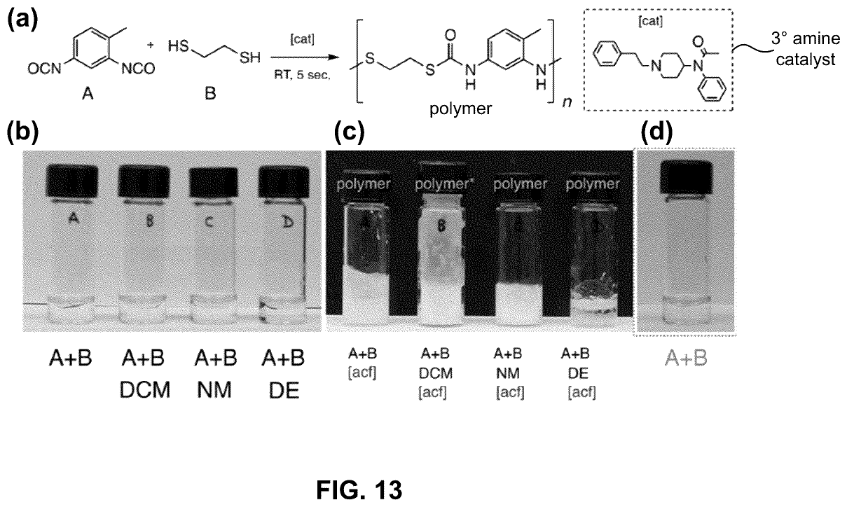

FIG. 13 illustrates a reaction of toluyl diisocyanate (A) and 1, 2-ethanedithiol (B) in the presence of catalytic amount of acetylfentanyl (˜ 1 mol. %) as a tertiary amine catalyst at ambient temperature, part (a). The reaction results in the formation of a polymer. In the image of part (b), vials A-D contain the mixture of both toluyl diisocyanate (A) and 1,2-ethanedithiol (B) and vials B-D include an added volume (˜20% of the total volume) of a solvent (DCM: dichloromethane; NM: nitromethane, and DE: diethyl ether). As shown in the image of part (c), addition of a catalytic amount of acetylfentanyl [acf], in a solution in dichloromethane, causes the formation of a polymer that is white translucent/cloudy solid within seconds (<5 seconds total time). The image of part (d) shows the control experiment that remains a colorless solution without the addition of the acetylfentanyl [acf].

Furthermore, the use of other amines, primary and secondary, may produce some polymerization but the mixture will remain predominately in liquid form. This side reaction may be minimized by using the same catalytic amounts of fentanyl that are used for comparative purposes, as the amines used in preliminary studies were used in ˜10 mol % relative to the bis-isocyanate and the 1,2-ethanedithiol.

### Direct Fluorescent Detection

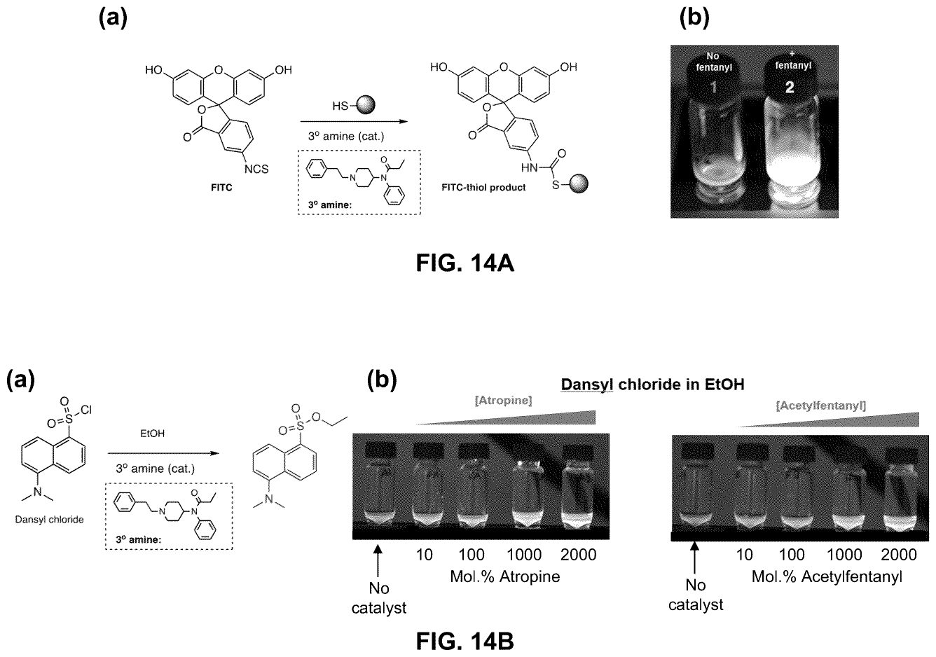

FIG. 14A illustrates a method of using fluorescence to detect the presence of a suspected target molecule. The reaction between a fluorescent dye fluorescein isothiocyanate (FITC) and ethane thiol in the presence of acetylfentanyl, a tertiary amine catalyst, is shown in part (a). The reaction at ambient temperature results in the formation of a fluorescent FITC-thiol product. The image of part (b) illustrates the product of the reaction. Vial 1 includes the reaction mixture without the catalyst acetylfentanyl, and vial 2 includes the reaction mixture with the catalyst acetylfentanyl. The presence of the tertiary amine catalyst acetylfentanyl demonstrates a remarkable fluorescence compared to the reaction mixture without the catalyst.

### Direct Fluorescent Detection Using a Modified Hinsberg Reaction

FIG. 14B illustrates another method of using fluorescence to detect the presence of a suspected target molecule. The reaction between a fluorescence molecule dansyl chloride and ethanol (EtOH) in the presence in the presence of a tertiary amine catalyst results in a change in fluorescence in the product, as shown in part (a). The presence of a tertiary amine catalyst in the reaction at ambient temperature results in a change in fluorescence of the dansyl chloride molecule. As illustrated in the image of part (b), two different tertiary amine catalysts (atropine and acetylfentanyl) cause a similar change in fluorescence in the dansyl chloride mixture. The original solution including the reaction mixture is fluorescent green (vial having No catalyst) and upon addition of catalysts, as illustrated with a natural occurring tertiary amine such as atropine and a synthetic tertiary amine such as acetylfentanyl, the green fluorescent color changes to a teal fluorescent color (shown in the image as a brighter fluorescence) when irradiated with UV light at λ=365 nm. The triangular bar above the vials containing the catalyst indicates the increasing concentration of catalyst having a tertiary amine (e.g., atropine, acetylfentanyl) added to the solution of dansyl chloride in ethanol.

### In Use

Potential use lies in the areas of detection of suspected target molecules (e.g., atropine, heroin, in different settings (airport security, clandestine laboratories, environmental setting, clinical setting, drug confiscation setting, etc.). The embodiments described herein would enable ready identification of contaminated surfaces both within and outside of a contaminated drug lab, spills at border crossings during search, etc. There are a number of applications that have been enumerated in the invention that can be commercialized.

The inventive concepts disclosed herein have been presented by way of example to illustrate the myriad features thereof in a plurality of illustrative scenarios, embodiments, and/or implementations. It should be appreciated that the concepts generally disclosed are to be considered as modular, and may be implemented in any combination, permutation, or synthesis thereof. In addition, any modification, alteration, or equivalent of the presently disclosed features, functions, and concepts that would be appreciated by a person having ordinary skill in the art upon reading the instant descriptions should also be considered within the scope of this disclosure.

While various embodiments have been described above, it should be understood that they have been presented by way of example only, and not limitation. Thus, the breadth and scope of an embodiment of the present invention should not be limited by any of the above-described exemplary embodiments but should be defined only in accordance with the following claims and their equivalents.

What is claimed is:

1. A method for detecting a suspected target molecule, the method comprising:
obtaining a reaction mixture that is not reactive at ambient temperature, the reaction mixture comprising at least two reactants;
adding the suspected target molecule to the reaction mixture for causing a reaction involving the reactants in the reaction mixture, the suspected target molecule acting as a catalyst for the reaction, wherein the suspected target molecule includes a tertiary amine; and
detecting presence of a product formed from the reactants in response to the addition of the suspected target molecule.

2. The method as recited in claim 1, wherein the suspected target molecule is an opioid molecule.

3. The method as recited in claim 2, wherein the opioid molecule is selected from the group consisting of: a synthetic opioid, an analog of a synthetic opioid, hydrocodone, codeine, morphine, oxycodone, and heroin.

4. The method as recited in claim 1, wherein the suspected target molecule is an alkaloid selected from the group consisting of: atropine, cocaine, and scopolamine.

5. The method as recited in claim 1, wherein the suspected target molecule is selected from the group consisting of: LSD, IP-LSD, and a derivative of LSD.

6. The method as recited in claim 1, wherein a first of the reactants includes a donor dye and a second of the reactants includes an acceptor dye, wherein the product includes a predefined fluorescent signal resulting from the reaction of the first reactant and the second reactant in the presence of the suspected target molecule.

7. The method as recited in claim 1, wherein the reactants include a thiol reactant and an isocyanate reactant.

8. The method as recited in claim 7, wherein the isocyanate reactant is a fluorescein isothiocyanate, wherein the product is a product having fluorescence.

9. The method as recited in claim 1, wherein the reactants include a nitroalkane reactant and an isocyanate reactant.

10. The method as recited in claim 1, wherein the reactants include an alcohol reactant and an isocyanate reactant.

11. The method as recited in claim 1, wherein the product causes a color change in the reaction mixture.

12. The method as recited in claim 1, wherein a substrate includes the suspected target molecule, wherein the reaction mixture is applied to the substrate, wherein the product forms on the substrate.

13. The method as recited in claim 1, wherein a substrate includes the reaction mixture, wherein the suspected target molecule is applied to the substrate, wherein the product forms on the substrate.

14. The method as recited in claim 13, wherein the substrate is selected from the group consisting of: an adhesive substrate, a mask, a swab, and a filter.

15. The method as recited in claim 1, wherein the reaction includes a polymerization reaction initiated by the presence of the suspected target molecule, wherein the product is a polymer.

16. The method as recited in claim 15, wherein the polymer is a polymer selected from the group consisting of: a polyurethane and a phosphorescent polymer.

17. The method as recited in claim 1, wherein the wherein the reaction occurs at ambient temperature.

18. The method as recited in claim 1, wherein the reaction mixture is present in a spray solution, the spray solution comprising a carrier solution, wherein the spray solution is configured to be sprayed onto the suspected target molecule.

19. The method as recited in claim 1, wherein the reaction mixture is used as a staining mixture for biological and/or molecular biology applications, wherein the staining mixture is applied to a sample selected from the group consisting of: a biological specimen and a biological product, wherein the product of the reaction causes a color change of portions of the sample if the suspected target molecule is present in the sample.

20. A product for detecting a suspected target molecule, the product comprising:
a substrate; and
a reaction mixture coupled to the substrate, the reaction mixture comprising at least two reactants, wherein the reaction mixture is not reactive at ambient temperature,
wherein the reaction mixture is configured to be reactive upon exposure thereof to a suspected target molecule that acts a catalyst, thereby causing the reaction mixture to react and form a product.

21. The product as recited in claim 20, wherein the substrate is selected from the group consisting of: a paper strip, an adhesive substrate, a mask, a swab, and a filter.

22. The product as recited in claim 20, wherein the reaction mixture is present on a surface of the substrate.

23. The product as recited in claim 20, wherein the reaction mixture is in the substrate.

24. A solution for detecting a suspected target molecule, the solution comprising:
a reaction mixture, the reaction mixture comprises at least two reactants; and
a solvent, wherein the reaction mixture is not reactive in the solvent at ambient temperature,
wherein the reaction mixture is configured to be reactive upon exposure to a suspected target molecule that acts as a catalyst, thereby causing the reaction mixture to react and form a product.

25. The solution as recited in claim 24, further comprising an additive, wherein the additive is selected from the group consisting of: a carrier, a second solvent, a component for enhancing a spray, and a combination thereof.
